(19) United States
(12) Patent Application Publication
(10) Pub. No.: US 2017/0023451 A1
(43) Pub. Date: Jan. 26, 2017

(54) Saliva-Derived Measures of Telomere Abundance and Sample Collection Device

(71) Applicant: TELOMERE DIAGNOSTICS, INC., Menlo, CA (US)

(72) Inventor: Calvin Harley, Murphys, CA (US)

(21) Appl. No.: 15/287,099

(22) Filed: Oct. 6, 2016

Related U.S. Application Data
(63) Continuation of application No. 14/370,005, filed on Jun. 30, 2014.
(60) Provisional application No. 61/582,261, filed on Dec. 31, 2011.

(51) Int. Cl. G01N 1/34; B01L 3/00; A61B 10/00; C12Q 1/68

(52) U.S. Cl. CPC G01N 1/34; C12Q 1/686; B01L 3/50825; A61B 10/0051; B01L 2300/0832; B01L 2200/0647; B01L 2300/042; B01L 2300/06

(57) ABSTRACT

This invention provides devices and methods for sample collection. Devices can include (a) a container having an opening and adapted to receive a liquid sample through the opening; (b) a cover configured to reversibly seal the opening; and (c) a capture device configured to be introduced into the container, wherein the capture device is configured to selectively bind cells of a first type and not to substantially bind cells of a second cell type. The sample can be analyzed to make a measure of telomere abundance and the abundance can be correlated to a measure of health.

### CROSS-REFERENCE TO RELATED APPLICATIONS

This application claims the benefit of the priority date of U.S. provisional patent application 61/582,261, filed Dec. 31, 2011, the contents of which are incorporated herein in their entirety.

### STATEMENT AS TO FEDERALLY SPONSORED RESEARCH

None.

### BACKGROUND OF THE INVENTION

Telomeres are structures at the ends of chromosomes characterized by repeats of the nucleotide sequence (TTAGGG)ₙ. Shortening of human telomeres, the protective “caps” at the ends of chromosomes, is a natural phenomenon of cellular aging (C. B. Harley et al., Nature, 1990, 345:458-460). In humans, shortening can be accelerated by genetic and environmental factors, including multiple forms of stress such as oxidative damage, biochemical stressors, chronic inflammation and viral infections (Epel, E. S. et al., Proc. Natl. Acad. Sci. USA, 2004, 101(49):17312-5). Telomere length provides a measure of stem cell and immune system senescence, and is predictive of longevity, disease risk, and the potential of the body to respond to certain drugs.

Telomeres become dysfunctional when they are critically short, triggering a DNA damage response that often culminates in loss of cell and tissue function, and ultimately a broad range of diseases and early mortality. Short telomeres have been linked to risk of cancer (Willeit, P. et al., JAMA, 2010, 304(1):69-75), various kinds of fibrosis (Wiemann et al., FASEB Journal, 2002, 16(9):935-982; Cronkhite, J. T., et al., Am. J. Resp. Crit. Care Med., 2008, 178:729-737), diabetes (Salpea, K. and Humphries, S. E., Atherosclerosis, 2010, 209(1):35-38), and numerous other conditions. In a growing number of studies, changes in telomere abundance or telomerase activity have also been correlated with disease risk or outcome (Bautista, C. V., et al., Colorectal Dis., 2010, Sep. 27; Panossian, L. A. et al., Neurobiol. Aging, 2003, 24(1):77-84).

Telomeres are unique in that they can be dynamically altered by telomerase, lifestyle, and environmental factors. Telomere shortening serves as a cumulative measure of these exposures, and in turn, the presence of short telomeres yields information about both disease risk and likely response to certain drugs and interventions. (Steer, S. E., et al., Ann. Rheum. Dis., 2007, 66(4):476-480; Njajou, O. T., et al., J. Gerontol. A. Biol. Sci. Med. Sci., 2009, 64(8):860-4; Brouilette et al., 2007).

Various methods have been developed for the measurement of telomere length in genomic DNA, including Southern blotting (Kimura, M. et al., Nature Protocols, 2010, 5:1596-1607), Q-FISH (Rufer, N. et al., Nat. Biotechnol., 1998, 16:743-747), flow FISH (Baerlocher, G. M. et al., Cytometry, 2002, 47:89-99), and qPCR (Cawthon, R. M., Nucleic. Acids Res., 2002, 30(10):e47). All of these methods can be used in a clinical setting to monitor health status and permit physicians to prescribe prophylactic or therapeutic intervention tailored to the needs of the individual patient.

Saliva is characterized as containing immune cells such as Langerhans cells, lymphocytes, neutrophils, granulocytes and non-immune cells, such as epithelial cells and bacterial cells. (See, e.g., Challacombc, S. J. and Naglik, J. R., Adv. Dental Res., 2006, 19:29-35; Nishita et al., BMC Medical Research Methodology, 2009, 9:71; Aps et al., Clinica Chimica Acta 321, 2002, 35-41). All of these cell types contain genomic DNA samples for which telomere length can be determined.

The following references refer to devices for saliva collection.

U.S. Pat. No. 5,910,122 (D'Angelo) refers to a saliva collector. According to D'Angelo, saliva samples are collected for body fluid constituent analysis by placing a sponge member into a patient's oral cavity. The sponge member is formed similarly to a pacifier nipple. Saliva is absorbed. The saliva is then expelled from the sponge member into a pipette. A filter may be placed between the sponge member and the pipette, through which the saliva is cleaned and molecular weight-selectively prepared by letting only substances through with a molecular weight below a cut-off weight. The integral unit is dismembered after the saliva has been transferred into the collection pipette, and the latter is tightly closed off for further handling.

U.S. Pat. No. 6,022,326 (Tatum et al.) refers to a method and device for automatic or semi-automatic collection of saliva has a mouthpiece on a wand. According to Tatum et al., the wand is connected to an interface section via a flexible conduit. Saliva is transported by aspiration into the device. Bulk air is removed and saliva is collected in a collection chamber. For collection of volatile components, air flow, vacuum, conduit diameter and length, and collection times are controlled and limited, to reduce loss of volatile components.

U.S. Pat. No. 7,482,116 (Birnboim) refers to compositions and methods for preserving and extracting nucleic acids from saliva. According to Birnboim, the compositions include a chelating agent, a denaturing agent, buffers to maintain the pH of the composition within ranges desirable for DNA and/or RNA. The compositions may also include a reducing agent and/or antimicrobial agent. Mentioned are methods of using the compositions of the invention to preserve and isolate nucleic acids from saliva as well as to containers for the compositions of the invention.

U.S. Pat. No. 8,221,381 (Muir et al.) refers to a container system for releasably storing a substance. According to Muir et al., the container system includes a vial having a sample storage chamber and a piercing member for piercing a membrane in the lid, which membrane seals a substance within a reservoir in the lid until the membrane is pierced by the piercing member. The container system optionally includes a funnel. There is also provided a method and kit for use of such a container system.

U.S. Patent Application 2004/0082878 (Baldwin et al.) refers to an oral fluid collection and transfer device that comprises a collection device and a test cartridge. According to Baldwin et al. the collection device includes a frame or chassis, and an absorbing pad for absorbing oral fluid and which is secured around part of the frame with part of the frame protruding from the pad. A collapsible cover covers the absorbing means and has apertures for the ingress of oral fluid into contact with the absorbing pad. A cap covers the part of the frame protruding from the absorbing pad. The cap and the cover latch together to surround the frame and the absorbing pad. The device also includes a fluid adequacy indicator in the form of an electrical circuit with an LED which is completed when the absorbing means has absorbed a predetermined volume of oral fluid. The test cartridge has a collection chamber to allow insertion of the collection device into the test cartridge. A test strip is used to for test the oral fluid for the presence of analytes. The collection device is located at a fixed location relative to the test cartridge within the collection chamber, in which location the absorbing pad undergoes a controlled degree of compression, thereby transferring a predetermined volume of oral fluid from the absorbing pad to the test strip.

U.S. Patent Application 2004/0082878 (Curry et al.) refers to a sample receiving device for releasably storing a substance. According to Curry et al. the sample receiving device includes a lid having a reservoir for retaining the substance, and a pierceable barrier sealing the substance within the reservoir; and b) a funnel for receiving a sample and configured for closure by the lid. The funnel is configured for releasable attachment to a sample receptacle such that a sample can be provided to the funnel and travel through the channel in the funnel into the sample receptacle. Further, the funnel includes one or more cutting ribs for cutting the pierceable barrier such that upon cutting of the pierceable barrier the substance is released from the reservoir, flows through the channel in the funnel and into the sample receptacle to be mixed with the sample. Mentioned is a kit for collecting and storing biomolecules.

The statements in the Background are not necessarily meant to endorse the characterization in the cited references.

### SUMMARY OF THE INVENTION

This invention provides a sample collection device, e.g. a saliva collection device, methods of use and methods of determining measures of telomere abundance and methods of correlating these measures with measures of health.

In one aspect, this invention provides a kit comprising: (a) a container having an opening adapted to receive a liquid sample through the opening; (b) a cover configured to reversibly seal the opening; and (c) a capture device configured to be introduced into the container, wherein the capture device is configured to selectively bind cells of a first type and not to substantially bind cells of a second cell type.

In one embodiment the capture device substantially binds at least one cell other than a lymphoid and/or myeloid cell.

In another embodiment the cells of a first type are targeted for removal and the cells of a second type are targeted for analysis.

In another embodiment the cells of the first type comprise epithelial cells and the cells of the second type comprise at least one of myeloid cells and lymphoid cells.

In another embodiment the cover is configured as a screw top or a snap top.

In another embodiment the cover is reversibly or irreversibly attached to the container.

In another embodiment the capture device comprises a wand.

In another embodiment the capture device binds a surface molecule on epithelial cells.

In another embodiment the surface molecule is Ep-CAM.

In another embodiment the capture device comprises particles comprising capture moieties that bind the target cells.

In another embodiment the container includes a first compartment and a second compartment, wherein: the first compartment and the second compartment are separated from one another by a breakable barrier; the first compartment is configured to receive a liquid sample from the opening in the container; the second compartment is closed and contains a cell lysis solution; and wherein the container is configured such that breaking the breakable barrier mixes a sample solution in the first compartment with the lysis solution in the second compartment.

In another embodiment the container comprises a vial having an open end and a closed end, wherein the first compartment is positioned closer to the open end and the second compartment is positioned closer to the closed end, and wherein the breakable barrier comprises a septum in the vial.

In another embodiment the capture device is configured as a wand comprising a collar, wherein the collar is configured to prevent a tip of the wand from piercing the breakable barrier when the wand is inserted into the opening of the container.

In another embodiment the container is configured such that manual squeezing of the container breaks the breakable barrier.

In another embodiment the first compartment comprises a non-denaturing solution.

In another embodiment the kit further comprises a container for the capture device.

In another embodiment the kit further comprises a second capture device comprising second binding moieties that bind cells other than epithelial cells and that do not substantially bind myeloid cells or lymphoid cells.

In another aspect this invention provides a method comprising: introducing saliva into a container; and removing epithelial cells from the saliva in the container, wherein at least one cell type selected from myeloid cells and lymphoid cells is retained in the saliva in the container.

In one embodiment saliva is introduced into the container by a subject by spitting, drooling or dripping from the mouth.

In another embodiment removing epithelial cells comprises introducing into the saliva in the container a capture device configured to bind epithelial cells, binding the epithelial cells to the capture device and removing the capture device with bound epithelial cells from the container.

In another embodiment the capture device comprises a binding agent that binds a surface molecule on epithelial cells.

In another embodiment the surface molecule is Ep-CAM.

In another embodiment the method further comprises: after removing the epithelial cells, lysing the retained cells in the container.

In another embodiment the container includes a first compartment configured to receive the saliva and a second compartment comprising a lysis solution, wherein the second compartment is separated from the first compartment by a breakable barrier and wherein lysing the retained cells comprises breaking the barrier to allow the lysis solution to mix with the saliva.

In another embodiment the method further comprises, before lysing the cells, sealing the container.

In another embodiment the method further comprises: removing bacterial cells from the saliva in the container.

In another embodiment the method further comprises: introducing the removed epithelial cells into a second container.

In another embodiment the method further comprises lysing the epithelial cells in the second container.

In another embodiment the method further comprises washing the removed epithelial cells before lysing the epithelial cells.

In another embodiment the method further comprises introducing into the saliva in the container a second capture device configured to bind second dells other than epithelial cells, binding the second cells to the second capture device and removing the second capture device with bound epithelial cells from the container.

In another embodiment the method further comprises: removing from the saliva in the container a subset of immune cells.

In another embodiment the method further comprises mixing the saliva in the container with a non-denaturing solution.

In another aspect this invention provides a method comprising: introducing saliva into a container; capturing epithelial cells in the saliva in the container with capture particles having moieties that bind epithelial cells, wherein at least one cell type selected from myeloid cells and lymphoid cells remains retains free in the saliva in the container.

In one embodiment the capture particles are magnetically responsive particles and the method further comprises sequestering the epithelial cells bounds to capture particles with magnetic force.

In another aspect this invention provides a method comprising determining a measure of telomere abundance on cells from a saliva sample, wherein the cells are substantially free of epithelial cells.

In another aspect this invention provides a kit comprising: (a) a container having an opening and adapted to receive a liquid sample; (b) a cover configured to reversibly seal the opening; and (c) capture particles having binding moieties that bind epithelial cells but that do not substantially bind at least one of myeloid cells and lymphoid cells.

In another aspect this invention provides a method comprising: determining a measure of telomere abundance in saliva-derived cells from a subject sample; and correlating the measure with: (1) a measure of health; (2) a risk of a pathological condition; (3) a telomeric disease or (4) drug responsiveness.

In one embodiment the method further comprises: introducing saliva into a container; and capturing epithelial cells in the saliva in the container with capture particles having moieties that bind epithelial cells, wherein at least one cell type selected from myeloid cells and lymphoid cells remains retains free in the saliva in the container as saliva-derived cells.

In another embodiment the subject sample comprises saliva enriched for lymphoid and/or myeloid cells.

In another embodiment the measure of telomere abundance is a measure of relative abundance.

In another embodiment the measure of relative abundance is abundance of telomeric DNA relative to abundance of total subject genomic DNA.

In another embodiment the measure of relative abundance is abundance of telomeric DNA relative to abundance of a genomic reference sequence.

In another embodiment the genomic reference sequence is a single copy reference nucleotide sequence (e.g., human beta-globin) or abundance of non-telomere repetitive DNA (e.g., Alu repeats or centromeric repeats).

In another embodiment the measure of telomere abundance is absolute abundance.

In another embodiment wherein absolute abundance is measured as length of telomeric sequences.

In another embodiment the determining a measure of telomere abundance comprises measuring average telomere length in the sample by qPCR, Southern blot, nucleic acid sequencing, in situ hybridization or flow FISH.

In another embodiment measurements are detected in relative fluorescence units (RFU) or by PicoGreen®.

In another embodiment saliva-derived cells are predominantly from one saliva gland.

In another embodiment the method further comprises correlating the measure with a measure of health.

In another embodiment the measure of health is a Health Status Survey score of perceived stress.

In another embodiment the risk of a pathological condition is a risk of disease.

In another embodiment the disease is a disease of aging.

In another embodiment the disease of aging is selected from the group consisting of cardiovascular disease, diabetes, cancer, liver fibrosis, and depression.

In another embodiment the disease of aging is cardiovascular disease and wherein a measure lower than average in a population correlates with or predicts increased risk of cardiovascular disease.

In another embodiment the method comprises correlating a measure of telomere abundance in the two or three lowest tertiles of a population with significantly higher risk for cardiovascular disease compared with a measure in a top tertile of the population.

In another embodiment the risk of the disease of aging is further correlated with the measure of telomere abundance determined from a blood sample.

In another embodiment, the population is age-matched with the subject.

In another embodiment the method comprises correlating the measure with a telomeric disease.

In another embodiment the telomeric disease is selected from the group consisting of Dyskeratosis Congenita, pulmonary fibrosis, aplastic anemia and interstitial pneumonia.

In another embodiment the method comprises correlating the measure with drug responsiveness.

In another embodiment the drug is selected from a statin, wherein short telomere length is correlated with drug responsiveness or imetelstat (GRN163L), wherein long telomere length is correlated with drug responsiveness.

In another embodiment the method further comprises reporting the correlation to the subject.

In another embodiment the method further comprises providing the subject with a diagnosis or a prognosis based on the correlation.

In another embodiment the method further comprises treating the subject based on the correlation.

In another aspect this invention provides a method for monitoring the status of a subject comprising: determining measures of telomere abundance from cells in each of saliva-derived subject sample in a plurality of saliva-derived subject samples taken over a period of time; b) determining differences in the measures; and c) correlating the differences with progression of a telomeric disease, wherein decreases in the measures indicates progression of the disease.

In another embodiment the method further comprises: introducing saliva into a container; capturing epithelial cells in the saliva in the container with capture particles having moieties that bind epithelial cells, wherein at least one cell type selected from myeloid cells and lymphoid cells remains retains free in the saliva in the container as saliva-derived cells.

In another aspect this invention provides a method comprising: a) sending a container comprising a subject sample comprising saliva; and b) receiving an electronically transmitted signal containing information indicating a determination of an measure of telomere abundance in the sample or a correlation of an measure of telomere abundance with: (1) a measure of health; (2) a risk of a pathological condition; (3) a telomeric disease or (4) drug responsiveness.

In another aspect this invention provides a method comprising: determining a rate of change in a measure of telomere abundance in saliva-derived cells from a plurality of subject samples, each sample taken at different times; and correlating the rate of change with: (1) a measure of health; (2) a risk of a pathological condition; (3) a telomeric disease or (4) drug responsiveness.

In one embodiment, the measure of telomere abundance is average telomere length or percentage of short telomeres.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a container of this invention containing saliva and having a capture device inserted into the saliva.

FIG. 2 shows a kit of this invention.

FIG. 3 lists antibodies available from Miltenyi Biotec (Auburn, Calif.) that are useful for binding cells found in saliva.

### DETAILED DESCRIPTION OF THE INVENTION

Unless otherwise specified, the term “or” is used herein in the inclusive.

1. Saliva as a Cell Source

Current methods for determining telomere length and correlating telomere abundance to health status utilize DNA obtained from circulating white blood cells obtained from venous blood draws or peripheral blood collection. Genomic DNA is carefully isolated from the nucleated cells in the blood sample to avoid contamination. White blood cells (leukocytes) consist of cells originating from the spleen, lymph nodes and thymus gland, with lymphocytes and neutrophils predominating.

Cells found in saliva are different from those found in blood. Cell phenotypes in saliva have not been well-characterized, although it is known that saliva is relatively enriched for non-immune epithelial cells as well as Langerhans cells, and for lymphocytes, neutrophils or granulocytes (Challacombe, S. J. and Naglik, J. R., Adv. Dental Res., 2006, 19:29-35). Langerhans cells are typically found in the skin or epidermis and the epithelial cells likely arise from salivary glands. Thus, it is unexpected that telomere length determined from saliva samples would have a strong correlation with that from blood samples.

In certain embodiments, a subset of cells in saliva are used for determinations of measures of telomere length and correlated with health conditions. In particular, samples enriched for cells of myeloid origin and/or cells of lymphoid origin are used for this purpose.

### 1.1 Saliva Collection Methods

Saliva is an attractive source of samples for telomere tests because saliva can be collected non-invasively, for example, with a simple spit kit that rapidly lyses cells and stabilizes the DNA nucleic acids, thus greatly simplifying collection and reducing the chances of variation in measurements due to contamination and variations in the pre-analytic handling of subject sample. Saliva samples may be self-collected at home or at work at any time of day, obviating the need for a subject to visit a medical facility. Unlike venous blood samples, saliva samples do not carry the risk of infection, do not cause patient discomfort and are very convenient, especially if multiple samples are desired. Compared to blood testing, saliva testing is also less expensive.

In an embodiment of the present invention, the subject spits into a cup, a vial or other suitable collection device. In this embodiment, telomere abundance is determined for genomic DNA derived from a mixture of cell types found in the saliva sample. Devices for the collection of undifferentiated saliva-derived cells are commercially available. For example, the Oragene® Genotek DNA kit (DNA Genotek Inc., Kanata, Ontario, Canada) contains a storage buffer that lyses the cells present and thus stabilizes the genomic DNA in the sample. Other devices are available from Norgen Biotek (Ontario, Canada) and Greiner Bio-One (Kremsmunster, Austria). Mother embodiments of the invention, described herein, the collection device is configured to allow removal or sequestration of certain cell types and the analysis of DNA from selected cell types.

In another embodiment of the present invention, whole saliva that pools on the floor of the mouth may be collected into an appropriate cup, using a passive drool technique. Saliva pooled in the mouth may be drooled down a straw into an appropriate vial or other collection device. Some individuals find it easier and more aesthetically pleasing to collect saliva by placing an absorbent device in the mouth. Such devices are commercially available, for example, the Salimetrics Oral Swab, which is made of an inert polymer shaped into a 30×10 mm cylindrical roll (Salimetrics, State College, Pa.). The swab is inserted into the mouth under the tongue until it is saturated. The saturated swab is then placed into a protective tub to prevent contamination before processing. The saliva sample may be removed from the swab by centrifugation of the storage tube for 15 minutes at 3000 to 3500 rpm to extract the saliva for DNA purification.

Saliva may be collected from different locations in the mouth or from a single saliva gland. Saliva is secreted by several different types of glands located in the mouth. Most saliva is secreted by three pairs of major glands located symmetrically on either side of the mouth: The parotid, the submandibular, and the sublingual gland. The parotid glands empty through the parotid ducts, which open into the cheeks adjacent to the second upper molars. Each submandibular gland empties into one long duct, the submandibular (or Wharton's) duct, which opens at the sublingual caruncle underneath the tongue. The openings from the two ducts are found just to either side of the frenulum. The front portion of each sublingual gland empties into the major sublingual duct. This duct sometimes opens adjacent to the submandibular duct, whereas in other individuals, it merges with the submandibular duct just before reaching the mouth. The rear portion of each sublingual gland empties through ten to twelve lesser sublingual ducts. These short ducts open directly upward in a row through the floor of the mouth along the sublingual fold, which runs obliquely from the sublingual caruncle off to either side.

The secretory units of the saliva glands are made up largely of two types of secretory cells: serous and mucous. These cells form globular or tubular clusters known as the acini. The acinar cells import water, salts, and various other components derived from plasma, such as lymphocytes, and combine them to produce saliva. Saliva glands also contain duct cells that deliver saliva to the mouth and move ions in and out of the salivary product to finalize its composition.

Saliva from all of the glands contains certain common components, but concentrations of certain other components, notably hormones, can vary significantly from one type of gland to another. One embodiment of the present invention employs DNA derived from saliva collected primarily or exclusively from a single salivary gland. Mixed spit samples are unsuitable for this use and saliva must be collected using an absorbent pad or swab device as described above. For example, parotid saliva may be collected by placing a device between the cheek and upper gum next to the second upper molar, where the parotid duct opens into the mouth. If the saliva flow is unstimulated, or is collected at a low flow time of day, the flow rate of saliva may be slow and the device must be left in place until it is saturated to ensure that an adequate sample is collected. Infants, small children, and mentally or physically impaired individuals may require assistance from another in order to place the collection device appropriately. Since the ducts from the different salivary glands have outlets into the oral cavity at known positions, enrichment of saliva from specific glands can be accomplished by placing collection devices such as absorbent pads or cloth cords at such positions.

In certain embodiments of the present invention, it is desirable to collect saliva at a specified time of day. For studies designed to establish population normals, saliva is optimally collected at the same time of day from all subjects, for example, between the hours of about 8:00 am and about 12:00 noon. Collecting saliva at a fixed time point also helps maintain consistency of saliva composition between and within individuals, especially in cross sectional and longitudinal studies. Saliva production in humans follows a circadian rhythm, with maximal flow occurring at about 5:00 am to about 6:00 am and again at about 5:00 pm to about 6:00 pm. One skilled in the art will appreciate that a subject's circadian rhythm may be affected by skewed sleep cycles and other lifestyle changes. Saliva sample collection may be timed to coincide with an individual's maximal saliva flow or at other times according to the disease state being studied.

2. Collection Devices

Certain devices of this invention include a container configured to receive a liquid sample, such as saliva, and a capture device configured to selectively or specifically bind at least one (e.g., one or a plurality) of first cell types (e.g., epithelial cells or bacterial cells). When the capture device is introduced into the container containing a liquid sample, it binds cells of the first type, but substantially does not bind cells of at least one of second types (“second cell types”) (e.g., myeloid cells and/or lymphoid cells). When the capture device is removed from the container or sequestered within the container, cells of the first type are separated from cells of the second type and the liquid is enriched for cells of the second type. After removal or sequestration of cells of the first type, the liquid sample can be stabilized (e.g. with denaturants) and stored, and/or it can be analyzed, or the cells of the first type can be similarly stabilized, analyzed, or stored. As will be described herein, the container can be further adapted to prepare the liquid solution or the cells in it for storage or analysis.

In one embodiment, the liquid sample comprises saliva, cells of the first type to be removed or sequestered include epithelial cells, specific subsets of immune cells, tumor cells, non-immune cells, other human cells (e.g., stem cells), or bacterial cells, and cells of the second type to be enriched include, for example, the remaining (predominantly) myeloid or lymphoid cells. In such embodiments, one can selectively measure a characteristic in the cells of the second type without contaminating measurements from cells of the first type originally in the same saliva sample. The characteristic to be measured can be, without limitation, a measure of telomere abundance (e.g., average telomere abundance), abundance of telomeres within specific size ranges, rate of change of telomere length, mRNA or shRNA expression levels, receptor levels, and abundance of other specific proteins. Certain methods of this invention enrich the saliva in the container for at least one cell type, which is selected from myeloid cells and lymphoid cells, relative to other cells. Accordingly, in one embodiment, the collection device of this invention allows one to selectively measure telomere abundance in myeloid cells or lymphoid cells in saliva without interfering measurements from epithelial cells or bacterial cells.

A sample collection device of this invention includes a container configured to receive a liquid sample and a capture device configured to selectively or specifically capture cells of at least one first type in a liquid sample. In some embodiments, the sample collection device includes a closure for the container. In some embodiments, the sample collection device removes substantially all cells other than myeloid and/or lymphoid cells in the sample.

### 2.1 Container

A sample collection device of this invention includes a container configured to receive a liquid sample. The container can be configured to contain a volume of liquid sample between about 50 microliters and about 10 milliliters, a volume between about 100 microliters and about 2 milliliters, or a volume between about 500 microliters and about 3 milliliters.

The container can have an elongate shape, such as that of a tube or a vial. It can be tapered, e.g., more narrow toward a closed end of the container. The end can be rounded.

The container can comprise a mouth or orifice that is wider than a void in the container in which the sample is held. For example, the container can have an aperture or opening having a funnel shape. The orifice of the container can be sufficiently wide to receive a saliva sample directly from the mouth of a subject, or it can be configured to receive a sample from a separate collection device (e.g. a saliva-soaked sponge or cotton swab, or other devices such as perforated brushes that collect saliva from the mouth).

The container can be made of any material suitable to hold a liquid sample. For example, the container can comprise a polymer, a metal or a glass. Polymers useful in this invention include, without limitation, polyethylene (e.g., low density polyethylene or high density polyethylene), polyethylene terephthalate (PETE), polyvinyl chloride (PVC) and polypropylene (PP). In certain embodiments, the container comprises a soft or pliable material. In such cases, the container can be squeezable, e.g., configured to be squeezed by manual pressure. Metals useful in the invention must be non-reactive with the saliva samples and non-corrosive and may optionally be coated with a suitable polymer to achieve non-reactivity. Glass containers of the invention may be similarly coated on the inside to achieve non-reactivity. Optionally, they may be coated on the outside with materials such as latex to add durability and discourage inadvertent breakage.

In certain embodiments, the container can include a liquid formulated to decrease viscosity of a sample, such as saliva, received into the container. Examples of such liquids are buffers such as phosphate buffered saline (PBS) or Tris-EDTA, with or without low levels of weak detergents (e.g., greater than about 1% or greater than about 0.1% of triton X-100, Nonidet P-40, triton X-114, or Brij-35), or enzymes that help degrade polymers in saliva other than DNA. Such a solution can be non-denaturing, such that cells are not ruptured by the solution. This allows further cell separation if necessary. In certain embodiments the container comprises a plurality of compartments, each compartment adapted to hold a liquid. The compartments can be fluidically isolated from one another. A first compartment can communicate with (e.g., be open to) an opening in the container configured to receive a liquid sample. A second compartment can be closed.

The container can be configured so that the compartments can be brought into fluid communication with one another. In one embodiment, the container includes a breakable barrier, such as a septum, that separates the container into two compartments. The septum can be made of a material that can be broken by applying mechanical pressure, for example, manual pressure applied by squeezing the container. The septum can be configured as a membrane. The material can be crackable or friable. The septum can be sealed along the inside walls of the container. The septum can be made of a frangible plastic material that is weaker than the material of the walls of the container, e.g., because it is thinner.

In another embodiment, the second compartment can be configured as a free enclosure in the container, such as a bubble.

In containers having a plurality of compartments, an open compartment (i.e., a compartment communicating with an opening in the container) can be configured to receive a liquid sample. A closed compartment can contain a reagent for preparing the sample for further analysis.

The container can include a lysis buffer for lysing cells in the sample. The lysis buffer can be contained in the second compartment. A lysis buffer can include, for example, a buffer and a detergent, or a buffer and a denaturant such as guanidinium hydrochloride. For example, a lysis buffer can include tris-HCl, EDTA, EGTA, SDS, deoxycholate, tritonX and/or NP-40 or stronger denaturants such as urea or guanidinium salts.

The container can include, e.g., in a closed compartment, a liquid contained a reagent to preserve biomolecules in the liquid sample, e.g., to preserve nucleic acids, such as DNA or RNA. Material to stabilize DNA can include denaturing enzymes which degrade protein (e.g. proteinase K), or denaturing agents, such as guanidinium salts or urea.

### 2.2 Closure

The container of this invention is configured to be closed after receiving a sample. The device can include a top, such as a cap or a lid, for this purpose. The top can be a screw top, or a snap top, e.g., a top that seals the container through a friction fit. The top can be detached from the body of the container. Alternatively the top can be attached to the container, for example, through a hinge or a flexible connector. For example, the container and the top can be comprised in a single piece. The container also can have a slide or zip lock for this purpose.

### 2.3 Capture Device

A sample collection device of this invention can include at least one capture device, each capture device configured to selectively bind cells of at least one of selected cell type. Such capture devices substantially bind cells of at least one first cell type but substantially do not bind cells of at least one second type. For example, a capture device that selectively binds epithelial cells may substantially not bind myeloid or lymphoid cells. In some embodiments a capture device binds at least 60% of the cells of the first type and not more than 20% of cells of the second type, at least 80% of first type cells and not more than 10% of second type cells or at least 90% of first type cells and not more than 5% second type cells. In certain embodiments the capture device has binding moieties having sufficient affinity and sufficient binding capacity to capture a majority of cells of the first type without capturing a significant number of cells of the second type. Requirements of the affinity and binding capacities will vary greatly depending upon properties of the capturing entity (e.g. an antibody or aptamer) and the properties of the captured entity, e.g., a specific receptor or protein on a cell. The ability to capture cells can be a function of the density of the binding target on the cell. That is, a cell having many copies of a binding partner on a cell surface can be captured with a binding device having less affinity for the partner than a cell with only a few copies, while a cell having fewer copies of a binding partner may need to be captured with a binding partner having greater affinity for that partner.

In one embodiment, the capture device comprises at least one solid support to which is attached at least one binding moiety that binds cells of interest. Unless otherwise specified, binding moiety and binding moieties refer to chemical entities of a single type, as distinguished from “different” binding moieties, which refers to chemical entities of different types.

### 2.3.1 Solid Support

Generally, a binding moiety is attached to a solid support. The solid support can comprise a single article or a collection of articles.

The single article can be configured to be introduced into and removed from the container, e.g., by hand or by an automated process. For example, the single article can have an elongate shape, such as a rod, wand or a stick. It can be relatively rigid, e.g., not floppy. It can be configured with an end that is wider or flatter than a handle of the article, for example as a paddle or spatula. The article can be configured with increased surface area for attaching binding moieties, such as including bristles or pores, or perforations, such as found in a porous pads or sponges. In some embodiments, at least part of the article can be configured similar to a pipe cleaner.

The solid support can comprise, without limitation, plastic, a biocompatible polymer, nitrocellulose, nylon, wood, ceramic or glass. Polymers useful in this invention include, without limitation, polyethylene (e.g., low density polyethylene or high density polyethylene), polyethylene terephthalate (PETE), polyvinyl chloride (PVC) and polypropylene (PP). Biocompatible co-polymers known to those skilled may also be used.

In another embodiment, the collection device can include a plurality of capture devices. That is, the capture devices can be configured as a collection or a plurality of articles. For example, the collection of articles can be a collection of elongate articles. Alternatively, the collection of articles can include a plurality of particles. An elongate article can have capture moieties attached substantially at an end of the article, or all along the article.

A sample collection device of this invention can include different combinations of binding moieties attached to different combinations of capture devices. For example, the collection device can include binding moieties of one type attached to at least one (e.g., one or a plurality of) solid support. The collection device can include a plurality of different types of binding moieties attached to the same solid support, and can include a plurality of these solid supports. The collection device can include a plurality of different binding moieties, each binding moiety attached to a different solid support. The collection device can have a plurality of different binding moieties, each directed against a single target molecule or against a single cell type.

In certain embodiments, the capture device is configured as one or a plurality of particles, such as beads, with the binding moiety covalently bonded to the bead using technology known in the art (e.g., U.S. Pat. No. 6,444,261; Plaskine et al.). Such particles can be sequestered from the liquid sample. For example, the particles can be magnetically responsive particles that can be sequestered by applying a magnetic force. If the cells to be analyzed are to be lysed in the container, then the particles with unwanted cells attached can be removed from the container before lysis.

This invention contemplates several different combinations of binding moieties and solid articles. This includes, for example, a single article having binding moieties against a single target (e.g., EpCAM) or a plurality of different binding moieties, each different binding moiety in the plurality against a different target (e.g., against EpCAM and one or more other epithelial cell surface antigens). Alternatively, a plurality of solid supports can each have binding moieties against a single target or a plurality of different binding moieties, each different binding moiety in the plurality against a different target. In one embodiment, a kit has a plurality of solid supports, each solid support having one or a plurality of different binding moieties directed against a single cell type. For example, a first solid support can have binding moieties directed against epithelial cells and a second solid support can have moieties directed against bacterial cells, e.g., antibodies that bind to bacterial surface antigens.

Binding moieties can be attached to solid supports by any means known in the art that leaves the binding portion of the moiety free to perform its capture function. The attachment means must be sufficiently robust to resist cleavage of binding moiety from the solid support during the capture process and subsequent manipulation of the bound cells. (See: Yoon, M. et al., J. Biomed. Sci. Engr., 2011, 4:242-247.) Typically, a chemical functional group distant from the binding portion of the capture moiety covalently couples with the solid support. For example, according to the method of Keogh, a 2-aminoalcohol functional group on the binding moiety may be oxidized with periodate to an aldehyde moiety which is reacted with an amine moiety on the surface of the solid support to form an imine moiety, and the imine moiety is then reduced to form an amine linkage immobilizing a coating of the binding moiety on the surface (U.S. Pat. No. 5,945,319). Alternatively, physical adsorption to, electrostatic attraction to or ionic linkages to solid support may be used. For example, ionic attachment of the binding moiety may be accomplished via a guanidino functional group (U.S. Pat. No. 5,928,916; Keogh).

The method of attaching the binding moiety to the solid support will be adapted to the type of solid support used. For example, if glass or ceramic is employed, it may be frosted according to the method Sugiura (U.S. Pat. No. 4,280,992), where the surface of the support is mechanically or chemically treated to produce a minutely uneven surface. A silane coupling agent including, for example, alkoxysilyl groups (such as methoxy- or ethoxy-substituted sylyl groups), halosilyl groups (such as chloro-substituted silyl groups), is reacted with the uneven glass surface. A cross-linking agent reactive with functional groups, such as amino, carboxyl and/or thiol group(s), on the binding moiety, then is reacted with the activated glass surface to form a covalent bond with the binding moiety. Groups suitable for cross-linking in this embodiment, as well in the embodiment where the solid support is a polymer, include, for instance, carboxyl, epoxy, haloalkyl (such as chloroethyl and chloropropyl), aldehyde, amino (primary and secondary amino), thiol, isocyanate, carboxylate, imino and nitrile (or cyano) groups, and the like. More specifically, examples of suitable functional groups reactive with the amino group are carboxyl, epoxy, haloalkyl and aldehyde groups; suitable functional groups reactive with the carboxyl group include, for example, amino (primary and secondary amino) and epoxy groups; and suitable functional groups reactive with the thiol group include thiol, epoxy, haloalkyl and aldehyde groups, and the like.

### 2.3.2 Binding Moiety

The binding moieties can be any material that selectively binds cells targeted for separation. Materials that selectively bind target cells include, for example, polypeptides (e.g., antibodies, receptors) or nucleic acids (e.g., aptamers). The binding moiety can be selected to bind to the target analyte with an affinity of at least 10⁻³ M, 10⁻⁴ M, 10⁻⁵M, 10⁻⁶ M, 10⁻⁷ M, 10⁻⁸ M, 10⁻⁹ M, 10⁻¹⁰ M, 10⁻¹¹ M or 10¹² M.

A binding moiety may be any protein that selectively or specifically binds a target of interest. Thus, a binding domain protein may be, for example, a receptor ligand, a ligand receptor or a ligand binding domain thereof (e.g., an extracellular domain (ECD) thereof), an antibody, or antigen-binding fragment thereof, a single-chain antibody, or antigen-binding fragment thereof.

The binding moiety can be an antibody. The term “antibody” refers to a polypeptide structure, e.g., an immunoglobulin, conjugate, or fragment thereof that retains antigen binding activity. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety (e.g., chimeric or bispecific antibodies or scFv's), and fragments, such as Fab, F(ab′)2, Fv, scFv, Fd, dAb and other compositions.

### 2.3.3 Target Cells

Capture devices of this invention selectively or specifically bind cells targeted for separation. In any sample that contains a plurality of cell types, the practitioner may desire to select certain cell types for analysis, or to separately analyze cells of different types. Such selection can involve removing cells of a certain type from a sample, and analyzing the cells retained in the sample or analyzing the cells removed from the sample. For example, it may be an object of investigation to analyze a characteristic of myeloid cells or lymphoid cells (e.g., to measure telomere abundance) in a sample, but not to also simultaneously analyze the characteristic in other cells of other types, such as epithelial cells or other human cells. Accordingly, binding moieties can be selected to bind and remove only unwanted cells types.

Immune cells are of particular interest in measures of telomere abundance. These include lymphoid cells and myeloid cells. In certain embodiments, lymphoid cells are the object of analysis. In certain embodiments, granulocytes are the object of interest. In another embodiment, naïve T cells (cd95 expressing cells) are the object of analysis.

The sample can be a saliva sample, e.g., a human saliva sample. Saliva contains several different cell types, including non-immune epithelial cells, cells of lymphoid origin (e.g., lymphocytes) and cells of myeloid origin (e.g., Langerhans cells neutrophils or granulocytes) and bacterial cells. In such a case, it may be desirable to separate one or more of these cell types from the other types. In one embodiment, binding moieties are selected to allow selective capture of epithelial cells while substantially not capturing cells of lymphoid or myeloid origin. In other embodiments, binding moieties are selected to selectively capture myeloid cells or lymphoid cells. Cells of a certain type can be captured by using binding moieties directed to molecules on the surface of the target cells that are not present on the surfaces of cells of a different cell type.

Epithelial cells have cell surface markers that differentiate them from myeloid and lymphoid cells. These markers include, without limitation, epithelial cell adhesion molecule (EpCAM, CD326), E-cadherin, Epithelial Membrane Antigen (EMA or MUC1) and pan cytokeratin. Specifically, epithelial cells display a cell surface protein referred to, variously, as epithelial cell adhesion molecule (“Ep-CAM”), tumor-associated calcium signal transducer 1 (“TACSTD1”), CD326 and 17-1A antigen. References that refer to molecules that bind Ep-CAM (e.g., anti-Ep-CAM antibodies) include: U.S. Pat. No. 7,557,190 (Barbosa et al.), U.S. Patent Application 2005/0009097 (Better et al.); U.S. Patent Application 2007/0274982 (Peters et al.), U.S. Patent Application 2010/0092491 (Anastasi et al.) and U.S. Patent Application 2010/0311954 (Chamberlain et al.).

Binding moieties chosen to separate epithelial cells from cells of different classes, such as lymphoid cells or myeloid cells, may include a variety of antibodies directed against these cell surface markers. A capture device can include binding moieties directed against one marker. A capture device can include a combination of binding moieties, each directed against a different marker. Compositions having antibodies directed against markers on epithelial cells are commercially available. For example, EMD Millipore (at world wide web URL millipore.com, Billerica, Mass., USA) commercializes several antibodies specific for epithelial cell surface markers. These include, for example, anti-EpCAM (e.g., MAB4444), Anti-Epithelial Specific Antigen Antibody (CBL251), Anti-Keratin Epithelial Antibody (MAB1611). Antibodies Online (at world wide web URL antibodies-online.com, Atlanta, Ga., USA) sells anti-EpCAM antibodies (e.g., ABIN361630). Thermo Scientific (at world wide web URL pierce-antibodies.com, Rockford, Ill. USA) sells anti-EpCAM (7E11) and anti-epithelial cell specific antigen (323/A3). Antibodies against EpCAM also are described in U.S. Pat. No. 7,557,190 (Barbosa et al.), U.S. Patent Application 2005/0009097 (Better et al.); U.S. Patent Application 2007/0274982 (Peters et al.), U.S. Patent Application 2010/0092491 (Anastasi et al.) and U.S. Patent Application 2010/0311954 (Chamberlain et al.).

In other embodiments, the capture device includes binding moieties directed against bacterial cells using antibodies specific for bacterial surface antigens.

3. Kits

Kits of this invention can be configured to provide a user with items for collecting a sample comprising a biological fluid, such as saliva, removing one or more types of cells from the fluid, optionally storing the removed cells, optionally lysing the cells remaining in the container, and transmitting at least the container and optionally the removed cells to a recipient, optionally by common carrier.

Accordingly, a kit of this invention can include a sample collection device of this invention and at least one other article. The kit can further comprise a shipping container adapted to hold the sample collection device and to be transmitted to a recipient. The kit also can contain a plurality of capture devices, each capture device configured to selectively capture a different cell type. The kit also can include at least one a holder, each configured to hold at least one capture device.

The shipping container can be any container suitable for shipping through a common carrier to a recipient. For example, the common carrier can be the United States Postal Service, FedEx or UPS. The shipping container can be, for example, an envelope, a box or a shipping tube. The shipping container can include padding configured to protect the contents from breakage risks commonly associated with the shipping method for which the shipping container is adapted.

In certain embodiments, one may wish to analyze a characteristic not only of the cells remaining in solution, but the cells removed from the solution. In this case, the kit can be provided with one or more separate containers configured to receive one or more capture devices. For example, the kit can contain one or more sleeves or bottles to accept an elongate article. In some embodiments, the elongate article can be attached to a container cover so that the elongate article can be sealed in the sleeve or bottle, for example by inserting the article into the container and snapping or screwing the cover to the container.

The kit also can contain instructions on how to use the kit. Instructions can include, for example, how to collect the biological sample, how to remove cells targeted for separation, how to lyse cells in the fluid, how to prepare materials for transport or how to transport the materials.

4. Methods

In methods of this invention, a biological fluid, e.g., saliva, is collected into a sample collection container. The amount collected can be between about 50 uL and about 2 ML, e.g., between about 100 uL and about 1 ML, about 50 uL, about 100 uL, about 400 uL, about 700 uL, about 1 mL, about 2 mL or greater than about 2 mL. A capture device having capture moieties is then put into contact with the fluid. The capture device remains in contact with the fluid for a time sufficient to capture a majority or substantially all of a cell type, e.g., epithelial cells, in the fluid slated for sequestration. Capture can be enhanced by moving the capture device through the liquid to improve contact between the binding moieties and the cells of interest. For example, the saliva can be stirred with the capture device. Then, the capture device is removed from the liquid, separating the captured cells from the cells of interest in the liquid. The capture device can be rinsed one or more times by swirling it in one or more separate containers which hold a buffer that helps remove non-specifically bound cells. The capture device can be discarded or saved for future use. The collection device is sealed with a cap. If the container also includes a lysis buffer in a separate compartment, the separation can be broken to bring lysis buffer in contact with the cells in the liquid. The sample collection container and, optionally, capture devices, can be enclosed within a shipping container and transmitted to a recipient, such as a laboratory, for processing.

### 4.1 Analysis

Cells collected and separated using a device or kit of this invention can be analyzed for any characteristic of interest. In certain embodiments, myeloid and lymphoid cells from saliva are analyzed to determine a telomere metric, such as a measure of telomere abundance or a rate of change of a measure of telomere abundance. Measures of telomere abundance include, for example, average telomere length, absolute telomere length or telomere length distribution.

Telomere lengths in human cells can vary from very short (e.g. <500 bp in length), to very long (e.g. >20 kbp in length). Kimura et al. describes telomere length distributions (Nature Protoc., 2010, 5(9):1596-607). One measure of telomere length distribution is the percentage of short telomeres, e.g., the percentage of telomeres less than 1 kb, less than 2 kb or less than 3 kb in length. For example, a sample can be tested to determine whether short telomeres constitute more than 15%, more than 20%, more than 25% or more than 30% of the total of telomeres. Recent studies suggest that the fraction of short telomeres may correlate better with measures of health than does average telomere length. Telomere length distribution can be determined by, for example, Southern blot, as described below.

Rate of change of a measure of telomere abundance also is correlated with measures of health. On average, telomeres shorten by about 50 bases per year. Rates of change greater than the average (e.g., at least 10% greater, at least 25% greater, at least 50% greater or at least 100% greater) are correlated with increased health risks, such as increased of cardiovascular disease. Rates of change slower than the average (e.g., less than 90% of this rate, less than 75% of this rate, less than 50% of this rate or increasing telomere length over time) are correlated with better health outcomes. Accordingly, a plurality of measures of telomere length can be determined over a given time period in order to determine the rate of change of telomere length. Such methods are discussed below in more detail.

Telomere abundance is correlated with many biological conditions and is a predictor of many measures of health. Accordingly, this invention provides methods for analyzing a subset of cell types in a biological sample for a characteristic of interest. In certain embodiments, this invention provides a method of analyzing lymphoid or myeloid cells in a saliva sample for a measure of telomere abundance. The method can include providing a saliva sample; separating at least one cell type targeted for removal from at least one cell type targeted for analysis; and analyzing the cell of the cell type targeted for analysis for a characteristic of interest. For example, cells targeted for separation can be a cell type that is not a lymphoid cell or a myeloid cell, for example, an epithelial cell or a bacterial cell. Cells targeted for analysis can be, for example, a lymphoid cell or a myeloid cell. Cells targeted for separation can be removed by, for example, contacting the sample with binding moieties that selectively bind the cell type or types target for separation, and that substantially do not bind to the cell type or types targeted for analysis, and sequestering the bound cells, e.g., by removing them from the liquid sample.

Samples comprising a subset of cells found in saliva can be used in measures of telomere abundance. The sample can be a saliva sample enriched for target cells found in saliva, such as myeloid cells (e.g., Langerhans cells, neutrophils or granulocytes) and/or lymphoid cells (e.g., lymphocytes such as naïve T cells). Measures of telomere abundance and rates of change of such measures indicate health, risk or existence of pathological conditions and responsiveness to drugs. Measures of telomere abundance of cells from such samples or rates of change of such measures can then be correlated with (1) a measure of health; (2) a risk of a pathological condition; (3) a telomeric disease or (4) drug responsiveness.

DNA is isolated from saliva samples by means known to one skilled in the art. In one embodiment, DNA may be robotically extracted from stabilized saliva samples using commercially available machines or using manual DNA purification kits, for example, a Puregene kit (Gentra Systems, Minneapolis, Minn.), as described by T. Rylander-Rudqvist et al. (Cancer Epidemiol. Biomarkers Prev., 2006, 15:1742-1745). Other commercially available DNA purification kits include the QIAamp DNA Mini Kit (Qiagen, Hilden, Germany) and the Norgen Saliva DNA Isolation Kit (Norgen Biotek, Ontario, Canada). The Norgen kit is based on spin column chromatography. Briefly, a saliva sample stabilized by treatment with a cell lysis agent is treated with a Proteinase K and a Purification Additive, followed by isopropanol. Binding Solution is added to the sample and the DNA is then bound to Norgen's BIND column, spun, washed to remove impurities and eluted with a buffer or water.

Other cells segregated from a sample (e.g., epithelial cells) can also be analyzed and used as independent diagnostic markers which will be more specific for the source organ of that cell type. For example, epithelial cells in a saliva sample arise from the salivary glandular epithelium or buccal epithelium and, hence, telomere length in these cells is expected to be more predictive of the health or disease status in the head and neck region.

### 4.2. Methods of Measuring Telomere Abundance

Measures of telomere abundance can be absolute or relative. Absolute measures of telomere abundance include, for example, total length of telomere sequences in a genome measured, for example, by number of nucleotides. More typically, telomere abundance is measured relative to a reference. Detection of telomere sequences can be measured in terms of signal strength produced in an assay. This signal strength can be compared with the signal strength produced by a reference sequence in the assay. The relative signal strength can function as a method of standardization. Standardized methods can be compared more easily between assays. For example, the signal produced by detection of telomere sequences can be compared with the signal produced by the measure of a sequence known to exist in the genome in single copy. One single-copy reference gene used for such purposes is the beta-hemoglobin gene. Thus, regardless of the assay method used, the relative signals of telomere sequences to reference sequences can be expressed, for example, as a ratio. This ratio can be used to compare results of telomere sequence abundance measurements.

### 4.3 qPCR

A variety of methods known in the art may be used in the present invention to determine average telomere length or telomere abundance. Preferably, the real time kinetic quantitative polymerase chain reaction (qPCR) is utilized as specifically modified for telomere length detection by Cawthon (Nucleic. Acids Res., 2002, 30(10):e47; U.S. Pat. No. 7,695,904). The method is simple and allows for rapid high throughput processing of large numbers of saliva-derived DNA samples. The qPCR method is based on the detection of the fluorescence produced by a reporter molecule which increases as the polymerase chain reaction proceeds. This increase in fluorescence occurs due to the accumulation of the PCR product with each cycle of amplification. These fluorescent reporter molecules include dyes that bind to the double-stranded DNA (for example, SYBR® Green or ethidium bromide) or sequence specific probes (for example, Molecular Beacons or TaqMan® Probes).

In the method of the present invention, primer probes specific to the repeated telomere sequence (TTAGGG)ₙ are used. The size of the primer may vary, in general, from 5 to 500 nucleotides in length, between 10 and 100 nucleotides, between 12 and 75 nucleotides, or between 15 to 50 nucleotides, depending on the use, required specificity, and the amplification technique. In the present invention, one embodiment utilizes a first primer which hybridizes to a first single strand of the target telomere sequence and a second primer which hybridizes to a second single strand of the target telomere sequence, where the first and second strands are substantially complementary. In this embodiment, for example, the paired primer set consisting of tel1 (5′-GGTTTTTGAGGGTGAGGGTGAGGGTGAGGGTGAGGGT-3′) [SEQ ID NO: 1] and tel2 (5′-TCCCGACTATCCCTATCCCTATCCCTATCCCTATCCCTA-3′) [SEQ ID NO: 2] may be used. In one embodiment, at least one of the primers comprises at least one altered or mutated nucleotide residue, which produces a mismatch between the altered residue and the 3′ terminal nucleotide of the other primer when the primers hybridize to each other. The presence of a mismatch at the 3′ terminal nucleotide blocks extension by polymerase, thus limiting non-target nucleic acid dependent extension reactions. In this embodiment, for example, the paired primer set consisting of tel 1b 5′-CGGTTTGTTTGGGTTTGGGTTTGGGTTTGGGTTTGGGTT-3′ [SEQ. ID No.: 3]; and tel 2b 5′-GGCTTGCCTTACCCTTACCCTTACCCTTACCCTTACCCT-3′ [SEQ. ID No.: 4] may be used. One skilled in the art will appreciate that other substantially complementary or mismatched sets of primers may be employed in this invention. Such primers are described in U.S. Pat. No. 7,695,904 (Cawthon et al.).

In hybridizing the primers to the telomere sequence and in the amplification reactions, the PCR assays are generally done under stringency conditions that allow formation of the hybrids in the presence of target nucleic acid. One skilled in the art can alter the parameters of temperature, salt concentration, pH, organic solvent, chaotropic agents, or other variables to control the stringency of hybridization and also to minimize hybridization of primers to non-specific targets. Following contact of the primers to the target telomere sequences, the reaction mixture is treated with a polymerase amplification enzyme. A variety of suitable polymerases are well known in the art, including, among others, Taq® polymerase (Invitrogen, Carlsbad, Calif.), KlenTaq™ (DNA Polymerase Technology, St. Louis, Mo.), Tfl polymerase (Promega, Madison, Wis.), and DynaZyme™ (Thermo Scientific, Lafayette, Colo.). Generally, although all polymerases are applicable in the present invention, polymerases are thermostable polymerases lacking 3′ to 5′ exonuclease activity since use of polymerases with strong 3′ to 5′ exonuclease activity tends to remove the mismatched 3′ terminal nucleotides.

In another aspect of the invention, various agents may be added to the reaction to increase performance of the polymerase, stabilize the polymerase from inactivation, decrease non-specific hybridization of the primers, and/or increase efficiency of replication. Such additives include, but are not limited to, dimethyl sulfoxide, formamide, acetamide, glycerol, polyethylene glycol, or proteinacious agents such as E. coli, single-stranded DNA binding protein, T4 gene protein, bovine serum albumin, and gelatin. In another aspect, one skilled in the art can use various nucleotide analogs for amplification of particular types of sequences, for example GC rich or repeating sequences. These analogs include, among others, c7-dGTP, hydroxymethyl-dUTP, diTP, and 7-deaza-dGTP.

Amplification reactions are carried out according to procedures well known in the art. Procedures for PCR are widely used and described (see for example, U.S. Pat. Nos. 4,683,195 and 4,683,202). In brief, a double stranded target nucleic acid is denatured, generally by incubating at a temperature high enough to denature the strands, and then incubated in the presence of excess primers, which hybridize (anneal) to the single-stranded target nucleic acids. A DNA polymerase extends the hybridized primer, generating a new copy of the target nucleic acid. The resulting duplex is denatured and the hybridization and extension steps are repeated. By reiterating the steps of denaturation, annealing, and extension in the presence of a second primer for the complementary target strand, the target nucleic acid encompassed by the two primers is exponentially amplified. The time and temperature of the primer extension step will depend on the polymerase, length of target nucleic acid being amplified, and primer sequence employed for the amplification. The number of reiterative steps required to sufficiently amplify the target nucleic acid will depend on the efficiency of the amplification. One skilled in the art will understand that the present invention is not limited by variations in times, temperatures, buffer conditions, and amplification cycles applied in the amplification process.

The products of the amplification are detected and analyzed by methods well known in the art. Amplified products may be analyzed following separation and/or purification of the products, or by direct measurement of product formed in the amplification reaction. For detection, the product may be identified indirectly with fluorescent compounds, for example, with ethidium bromide or SYBR™ Green, or by hybridization with labeled nucleic acid probes. Alternatively, labeled primers or labeled nucleotides are used in the amplification reaction to label the amplification product. The label comprises any detectable moiety, including fluorescent labels, radioactive labels, electronic labels, and indirect labels such as biotin or digoxigenin.

Instrumentation suitable for conducting the qPCR reactions of the present invention are available from a number of commercial sources (ABI Prism 7700, Applied Biosystems, Carlsbad, Calif.; LightCycler™ 480, Roche Applied Science, Indianapolis, Ind.; Eco Real-Time PCR System, Illumina, Inc., San Diego, Calif.; RoboCycler 40, Stratagene, Cedar Creek, Tex.).

When real time quantitative PCR is used to detect and measure the amplification products, various algorithms are used to calculate the number of target telomeres in the samples. (For example, see ABI Prism 7700 Software Version 1.7; Lightcycler Software Version 3). Quantitation may involve use of standard samples with known copy number of the telomere nucleic acids and generation of standard curves from the logarithms of the standards and the cycle of threshold (CO. In general, Cₜ is the PCR cycle or fractional PCR cycle where the fluorescence generated by the amplification product is several deviations above the baseline fluorescence. Real time quantitative PCR provides a linearity of about 7 to 8 orders of magnitude, which allows measurement of copy number of target telomere nucleic acids over a wide dynamic range. The absolute number of target nucleic acid copies can be derived from comparing the Cₜ values of the standard curve and the samples.

The amplified products are quantitated as described above. In an embodiment, real time quantitative PCR is used to determine the copy number of the telomere repetitive units, or telomere abundance, in the target nucleic acid sample. Standards for determining and comparing telomere repetitive unit number include use of single copy genes (for example, human beta-globin or ribosomal phosphoprotein 364) or a target nucleic acid of known copy number (e.g., a plasmid with a known number of telomere repetitive units). Additionally, the abundance of non-telomere repetitive DNA nucleic acid, for example, Alu repeats or centromeric repeats, may be used. Thus, a measure of relative telomere abundance is determined by the abundance of telomeric DNA relative to the abundance of a genomic reference sequence. By the methods described herein, the copy number of repetitive units of a large number of samples may be quantitated for purposes of determining the number of telomere repetitive units, and thus the average length of telomeres.

In an embodiment of the invention for the determination of telomere abundance by qPCR, the assay consists of two separate PCR reactions that are compared. A T (telomic) PCR value for subject saliva sample DNA is obtained along with an S (single copy gene) length for human beta-globin. The T value is divided by the S value to give a ratio that determines the length of a sample telomere relative to the abundance of genomic DNA as reflected by the S value. This ratio is thus proportional to the average telomere length per genome. The quantity of telomere repeats in each saliva-derived sample is a measured as the level of dilution of an arbitrarily chosen reference DNA sample that would make the experimental and reference samples equivalent with regard to the number of PCR cycles needed to generate a given amount of telomere PCR product during the exponential phase of PCR amplification. Similarly, the relative quantity of the single copy gene in each sample is expressed as the level of dilution of the referenced DNA sample needed to match it to the experimental sample with regard to the number of PCR cycles needed to generate a given amount of single copy gene PCR product during the exponential phase of the PCR. For each subject sample, the ratio of these dilution factors is the relative telomere to single copy gene (T/S) ratio. Therefore, T/S=1 when the unknown DNA sequence is identical to the reference DNA in its ratio of telomere repeat copy number to single copy gene copy number. The reference DNA sample may be from a single individual or may be derived from a pooled sample obtained from multiple subjects. The T/S ratio of one individual relative to the T/S ratio of another corresponds to the relative telomere lengths of their DNA.

### 4.4 Other Methods

Average telomere length may be measured by other methods known in the art. Such methods include, but are not limited to, direct nucleic acid sequencing, Southern blotting, in situ hybridization, and flow FISH.

Conventional techniques for the direct determination of nucleic acid sequences in isolated DNA may be employed in the present invention. For example, see: “DNA Sequencing,” The Encyclopedia of Molecular Biology, J. Kendrew, ed., Blackwell Science Ltd., Oxford, U K, 1995, pp. 283-286. Dye-terminator automated sequencing is now most commonly used for nucleic acid sequencing (“DNA Sequencing”, Lab Manager, at world wide web URL labmanager.com/?articles.view/articleNo/3364/article/DNA-Sequencing). Automated sequencing equipment is conveniently used and may be purchased from companies such as Applied Biosystems, Roche Applied Science, and Illumina Inc. Once the sequence of a DNA sample is determined, the number of copies of the telomere nucleotide sequence (TTAGGG) at either end can then be counted. This method of the present invention provides a measure of absolute telomere abundance by direct measurement of telomeric sequences.

Southern blotting (Southern, E. M., J. Mol. Biol., 1975, 98(3): 503-517) may also be utilized in the present invention to determine telomere abundance by detecting the specific presence of the human telomere nucleotide sequence (TTAGGG)ₙ. In the present invention, Terminal Restriction Fragment (TFR) Southern blotting combines the transfer of DNA fragments separated by electrophoresis to a filter membrane, followed by detection of the fragments by hybridization to probes specific for the (TTAGGG) sequence (Allshire, R. C. et al., Nucleic Acids Res., 1989, 17, 4611-4627). Such probes have sequences complementary to the telomere sequence. For ease of detection, probes are radioactively labeled or tagged with a fluorescent or chromogenic dye. The amount of radioactivity or fluorescence present may then be quantified to give the telomere abundance in the sample. M. Kimura et al. (Nature Protocols, 2010, 5:1596-1607) describes an appropriate Southern blot procedure for determining telomere length.

In situ hybridization may be used in the present invention. In situ hybridization is utilized to detect and locate the (TTAGGG)ₙ sequence in tissues or on chromosomes. A probe labeled with a radioactive or a fluorescent tag is applied to fixed tissues or chromosome preparations, where it hybridizes with any complementary sequences present. After unhybridized probe is washed away, the labeled probes reveal the location of the telomere sequences. The amount of radioactivity or fluorescence present may then be quantified and correlated to telomere length.

In the above embodiments of the present invention, fluorescence may be measured in relative fluorescence units (RFU). Fluorescence is detected using a charged coupled device (CCD) array, when the labeled fragments, which are separated within a capillary by using electrophoresis, are energized by laser light and travel across the detection window. A computer program measures the results, determining the quantity or size of the telomere-containing fragments, at each data point, from the level of fluorescence intensity (“Relative fluorescence unit (RFU)”, DNA.gov: Glossary, April 2011, world wide web URL dna.gov/glossary/). Samples that contain higher quantities of amplified DNA will have higher corresponding RFU values (Gertsch J. et al., Pharm Res., 2002, 19:1236-1243). An “RFU peak” is a relative maximum point along a graph of the analyzed data. In the present invention, it is important to normalize the resulting data to laboratory standards so that well-quantified results are used in desired clinical correlations.

Fluorescent in situ hybridization may be combined with flow cytometry in a technique known as flow fluorescence in situ hybridization (flow FISH). In the determination of telomere abundance in the present invention, flow FISH quantifies the number of copies of the (TTAGGG) sequence in genomic DNA isolated from saliva. Protocols for flow FISH and automated flow FISH have been standardized and published (G. M. Baerlocher et al., Cytometry, 2002, 47:89-99; G. M. Baerlocher and P. M. Lansdorp, Cytometry, 2003, 55:1-6; G. M. Baerlocher et al., Nature Protocols, 2006, 1(5): 2365-2376).

Briefly, flow FISH employs peptide nucleic acid probes of, for example, a 3′-CCCTAACCCTAACCCTAA-5′ [SEQ ID No.: 5] sequence labeled with a fluorescent probe, for example, fluorescin, to stain telomeric repeats hybridized with prepared DNA samples. Appropriate fluorescent probes are commercially available (eBioscience, San Diego, Calif.; Applied Biosystems, Carlsbad, Calif.; Miltenyi Biotech, Auburn, Calif.). The fluorescence yielded by probe staining is quantitative because the probe binds preferentially to DNA under the hybrization conditions. The DNA duplex cannot reform once it has been melted and annealed to the probe, allowing the probe to saturate its target repeat sequence (as it is not displaced from the target DNA by competing anti-sense DNA on the complementary strand), thus yielding a reliable and quantifiable readout of the frequency of the probe target after washing away of unbound probe. Fluorescence is measured using a flow cytometer, which has been appropriately calibrated. Flow cytometers are commercially available, from suppliers such as Millipore (Billerica, Mass.), Applied Biosystems (Carlsbad, Calif.), and BD Biosystems (Franklin Lakes, N.J.).

5. Conditions Correlated with Telomere Abundance

Telomere length determined from genomic DNA purified from blood samples has been shown to correlate with several important biological indices. These indices include, for example, chronological age, body-mass index, hip/weight ratio, perceived stress and cardiovascular risk. One measurement of telomere length is the telomere/single copy (“T/S”) ratio. It has been discovered that the T/S ratio determined from genomic DNA derived from saliva samples is highly correlated (a 0.7 Pearson correlation coefficient) with the T/S ratio determined by standard methods from blood samples. Such ratios in a given population can be divided into quantiles, for example, into tertiles. It has been found that individuals with telomere abundance in the lower two tertiles are at significantly higher risk for cardiovascular disease than those in the top tertile for telomere length.

In general, percentile value of measure of telomere abundance, e.g., T/S, in a population correlates with risk of disease and measures of health, with lower percentile scores correlating positively with decreased measures of health, increased disease risk and presence of telomere disease.

In a population, telomere length decreases with age. Accordingly, measures of telomere length for an individual can be compared with measures for persons in the same age range in the population, that is, an age-matched population. For example, a person at age 30 might have a measure of telomere abundance about equal to the population average for age 30, or equal to the population average for age 20 or age 40. Correlations of a measure of telomere abundance with measures of health are more accurate when compared with the measure for an age-matched population. The range for an age matched population can be, for example, one year, two years, three years, four years, 5 years, 7 years or 10 years.

### 5.1 Measures of Health

Telomere abundance determined from subject saliva-derived samples by the method of the present invention may be correlated with measures of health. The saliva-derived samples can be cells selected from saliva samples, e.g., lymphoid or myeloid cell. Of particular interest are measures of health involving perceived stress. Telomere shortening can be accelerated by genetic and environmental factors, including multiple forms of stress such as oxidative damage, biochemical stressors, chronic inflammation and viral infections (Epel, 2004, ibid.).

A convenient measure of general health status is the SF-36® Health Survey developed by John Ware (see, e.g., world wide web URL sf-36.org/tools/SF36.shtml). The SF-36 is a multi-purpose, short-form health survey with only 36 questions to be posed to patients, preferably by trained individuals. It provides an 8-scale profile of functional health and well-being scores as well as psychometrically-based physical and mental health summary measures and a preference-based health utility index. The survey is a generic measure, as opposed to one that targets a specific age, disease, or treatment group. Accordingly, the SF-36 has proven useful in surveys of general and specific populations, comparing the relative burden of diseases, and in differentiating the health benefits produced by a wide range of different treatments. In addition to the standard SF-36 survey, other specialized survey versions are available from the Medical Outcomes Trust (Hanover, N.H.) including SF-36v2™ Health Survey, SF-12® Health Survey, SF-12v2™ Health Survey, SF-8™ Health Survey, and SF-10™ Health Survey for Children. The SF-36 survey is used to estimate disease burden and compare disease-specific benchmarks with general population norms. The most frequently studied diseases and conditions include arthritis, back pain, cancer, cardiovascular disease, chronic obstructive pulmonary disease, depression, diabetes, gastro-intestinal disease, migraine headache, HIV/aids, hypertension, irritable bowel syndrome, kidney disease, low back pain, multiple sclerosis, musculoskeletal conditions, neuromuscular conditions, osteoarthritis, psychiatric diagnoses, rheumatoid arthritis, sleep disorders, spinal injuries, stroke, substance abuse, surgical procedures, transplantation and trauma (Turner-Bowker et al., SF-36® Health Survey & “SF” Bibliography: Third Edition (1988-2000), QualityMetric Incorporated, Lincoln, R.I., 2002). One skilled in the art will appreciate that other survey methods of general health status, for example, the RAND-36, may find use in the present invention.

The results from the SF-36 family of surveys are evaluated on a numerical scale. For example, data from the SF-36 surveys may be conveniently analyzed using commercially available software, for example, QualityMetric Health Outcomes™ Scoring Software 2.0 (QualityMetric Health Outcomes Solutions, Eden Prairie, Minn.).

In one embodiment of the present invention, subject saliva samples are collected over time and measurements of telomere abundance are determined from the samples. Appropriate time periods for collection of a plurality of samples include, but are not limited to, 1 month, 3 months, 6 months, 1 year, 2 years, 5 years and 10 years (for example, the time between the earliest and the last sample can be about these time periods). This method allows for monitoring of patient efforts to improve their general health status and/or to monitor their health status and/or disease risk. Since telomere length decreases with age, a finding that TL is maintained or increases with time within an individual indicates a health improvement, while loss of TL over time represents a decrease or worsening in health. In addition, subjects within the lower one or two tertiles of TL have been shown to be at increased risk of cardiovascular disease or cancer (Brouilette et al., 2007; Willeit et al., 2010) relative to the highest tertile group (the “reference population”). In the Willeit study, subjects in the lowest tertile were at greater risk of disease than those in the middle tertile. These general trends can be further quantified in terms of the observed T/S ratio.

In several studies using the qPCR technology for T/S determination, the tertile boundaries have been found to be roughly 0.9 and roughly 1.3, indicating subjects with T/S less than 0.9, or 0.9-1.3, or greater than 1.3 are relatively speaking at high, moderate, and low risk of disease. A T/S ratio value less than roughly 0.5 has been associated with “telomere disease”, a condition of very high disease risk due to accelerated telomere loss from inactivating mutations in telomerase or a telomere-related protein. Telomere disease is often associated with loss of stem cell regeneration capacity in the highly proliferative tissues such as skin, bone marrow, lung, liver, and gut. These values can be applied to all of the risk of diseases described below. The appropriate medical intervention that a doctor might perform for individuals with short telomeres but no clinical symptoms would typically be more frequent testing using conventional disease biomarkers for early diagnosis of specific diseases, or more aggressive treatment for subjects with clinical symptoms. In addition, since short telomeres have been associated with drug response (e.g. Brouilette et al., 2007; Rattain et al., 2008), the doctor might select a specific type of drug or a specific dose depending upon telomere length.

In certain embodiments of the invention, in addition to taking a measure of telomere abundance in a sample, one also can determine the amount of bacterial cells in the cells. Such bacterial cells will have been separated from the sample before testing. Bacterial cells in a sample can be measured, for example, by qPCR. High levels of bacterial DNA can lead to false telomere length measurements by interfering with qPCR. Accordingly, a sample can be rejected for analysis if bacteria, e.g., bacterial DNA levels, exceed a certain threshold (e.g., >600 ng/ul or >700 ng/ul.

### 5.2 Risk of a Pathological Condition

### 5.2.1 Diseases

Measuring the number of repetitive units of telomeres has a wide variety of applications in medical diagnosis, e.g., for disease risk, disease prognosis, and therapeutics. In particular, measurement of telomere length finds application in assessing pathological conditions leading to the risk of disease. In one embodiment of the invention, the disease is one associated with aging, for example but not limited to, cardiovascular disease, diabetes, cancer, liver fibrosis, and depression.

In one embodiment, the present invention finds use in the assessment and monitoring of cardiovascular disease. Telomere length in white blood cells has been shown to be shorter in patients with severe triple vessel coronary artery disease than it is in individuals with normal coronary arteries as determined by angiography (Samani, N. J. et al., Lancet, 2001, 358:472-73), and also in patients who experiencing a premature myocardial infarction before age 50 years as compared with age- and sex-matched individuals without such a history (Brouilette S. et al., Arterioscler. Thromb. Vasc. Biol., 2003, 23:842-46). Brouilette et al. (Lancet, 2007, 369:107-14) has suggested that shorter leucocyte telomeres in people prone to coronary heart disease could indicate the cumulative effect of other cardiovascular risk factors on telomere length. Increased oxidative stress also contributes to atherosclerosis, and increased oxidant stress has been shown to increase rates of telomere attrition in vitro (Harrison, D., Can. J. Cardiol., 1998, 14(suppl D):30D-32D; von Zglinicki, T., Ann. N. Y. Acad. Sci., 2000, 908:99-110). In cross-sectional studies, smoking, body-mass index, and type 1 diabetes mellitus have also been reported to be associated with shorter leucocyte telomere length (Valdes, A., et al., Lancet, 2005, 366:662-64; Jeanclos, E. et al., Diabetes, 1998, 47:482-86). Increased life stress, a factor known to increase the risk of coronary heart disease, has been shown to be associated with shorter telomeres, possibly as a consequence of increased oxidative stress (Epel, E. S. et al., Proc. Natl. Acad. Sci. USA, 2004, 49:17312-15.) Thus, smokers and patients with a high body-mass index, diabetes and/or increased life stress would all benefit from determination and continued monitoring of their telomere abundance according to the method of the invention.

Type 2 diabetes is characterized by shorter telomeres (Salpea, K. and Humphries, S. E., Atherosclerosis, 2010, 209(1):35-38). Shorter telomeres have also been observed in type 1 diabetes patients (Uziel O. et al., Exper. Gerontology, 2007, 42:971-978). The etiology of the disease in type 1 diabetes is somewhat different from that in type 2, although in both cases, beta cell failure is the final trigger for full-blown disease. Telomere length is thus a useful marker for diabetes since it is associated with the disease's progression. Adaikalakoteswari et al. (Atherosclerosis, 2007, 195:83-89) have shown that telomeres are shorter in patients with pre-diabetic impaired glucose tolerance compared to controls. In addition, telomere shortening has been linked to diabetes complications, such as diabetic nephropathy (Verzola D. et al., Am. J. Physiol., 2008, 295:F1563-1573), microalbuminuria (Tentolouris, N. et al., Diabetes Care, 2007, 30:2909-2915), and epithelial cancers (Sampson, M. J. et al., Diabetologia, 2006, 49:1726-1731) while telomere shortening seems to be attenuated in patients with well-controlled diabetes (Uziel, 2007, ibid.). The method of the present invention is particularly useful in monitoring the status of pre-diabetic and diabetic patients to provide an early warning for these complications and others.

The present invention is useful for determining telomere lengths of various types of cancer cells because activation of telomerase activity is associated with immortalization of cells. While normal human somatic cells do not or only transiently express telomerase and therefore shorten their telomeres with each cell division, most human cancer cells typically express high levels of telomerase and show unlimited cell proliferation. High telomerase expression allows cells to proliferate and expand long term and therefore supports tumor growth (Roth, A. et al., in Small Molecules in Oncology, Recent Results in Cancer Research, U. M. Martens (ed.), Springer Verlag, 2010, pp. 221-234). Shorter telomeres are significantly associated with risk of cancer, especially cancers of the bladder and lung, smoking-related, the digestive system and the urogenital system. Excessive telomere shortening likely plays a role in accelerating tumor onset and progression (Ma H. et al., PLoS ONE, 2011, 6(6): e20466. doi:10.1371/journal.pone.0020466). Studies have further shown that the effect of shortened telomeres on breast cancer risk is significant for certain population subgroups, such as premenopausal women and women with a poor antioxidative capacity (Shen J., et al., Int. J. Cancer, 2009, 124:1637-1643). In addition to the assessing and monitoring cancers in general, the present invention is particularly useful for the monitoring of oral cancers as it utilizes genomic DNA derived from saliva samples.

Cirrhosis of the liver is characterized by increasing fibrosis of the organ often associated with significant inflammatory infiltration. Wiemann et al. (FASEB Journal, 2002, 16(9):935-982) have shown that telomere shortening is a disease- and age-independent sign of liver cirrhosis in humans. Telomere shortening is present in cirrhosis induced by viral hepatitis (chronic hepatitis A and B), toxic liver damage (alcoholism), autoimmunity, and cholestasis (PBC and PSC); telomeres are uniformly short in cirrhosis independent of the age of the patients. Telomere shortening and senescence specifically affect hepatocytes in the cirrhotic liver and both parameters strongly correlate with progression of fibrosis during cirrhosis. Thus, the method of the present invention finds use in diagnosing and monitoring liver fibrosis.

Depression has been likened to a state of “accelerated aging,” and depressed individuals have a higher incidence of various diseases of aging, such as cardiovascular and cerebrovascular diseases, metabolic syndrome, and dementia. People with recurrent depressions or those exposed to chronic stress HI exhibit shorter telomeres in white blood cells. Shorter telomere length is associated with both recurrent depression and cortisol levels indicative of exposure to chronic stress (Wikgren, M. et al., Biol. Psych., 2011, DOI: 10.1016/j.biopsych.2011.09.015). However, not all depressed individuals show shortened telomeres equally because of a large variance in depressive episodes over a lifetime. Those who suffered from depression for long durations have significantly shorter telomeres due to longer exposure to oxidative stress and inflammation induced by psychological stress when compared with control populations (Wolkowitz et al., PLos One, 2011, 6(3):e17837). Thus, the method of the present invention may find use in monitoring depression.

### 5.2.2 Other Pathological Conditions

The present invention also finds use in diagnosis of diseases related to early onset of aging. For example, individuals with Hutchinson Gilford progeria disease show premature aging and reduction in proliferative potential in fibroblasts associated with loss of telomeric length (Allsopp, R. C. et al, Proc. Natl. Acad. Sci. USA, 1992, 89:10114-10118). Amplification and quantitation of the number of telomeric repeats according to the method of this invention is useful for determining disease risk or prognosis and taking appropriate interventional steps as described above.

### 5.3 Telomere Diseases

In one embodiment of the present invention, both the presence and the progress of telomeric-specific diseases may be determined using saliva-based samples. Telomeric diseases originate from defects in telomerase activity. Telomerase is a ribonucleoprotein complex required for the replication and protection of telomeric DNA in eukaryotes. Cells lacking telomerase undergo a progressive loss of telomeric DNA that results in loss of viability and a concomitant increase in genome instability. These diseases may be inherited and include certain forms of congenital aplastic anemia, in which insufficient cell divisions in the stem cells of the bone marrow lead to severe anemia. Certain inherited diseases of the skin and the lungs are also caused by telomerase defects. For telomere diseases, a threshold for T/S<0.5 is appropriate for some conditions. Also, a commonly used metric is an age-adjusted percentile telomere score less than <10% or preferably <1% relative to a normal population.

Dyskeratosis congenita (DKC), also known as Zinsser-Engman-Cole syndrome, is a rare, progressive bone marrow failure syndrome characterized by mucocutaneous abnormalities: reticulated skin hyperpigmentation, nail dystrophy, and oral leukoplakia (Jyonouchi S. et al., Pediatr. Allergy Immunol., 2011, 22(3):313-9; Bessler M., et al., Haematologica, 2007, 92(8):1009-12). Evidence exists for telomerase dysfunction, ribosome deficiency, and protein synthesis dysfunction in this disorder. Early mortality is often associated with bone marrow failure, infections, fatal pulmonary complications, or malignancy. The disease is inherited in one of three types: autosomal dominant, autosomal recessive, or the most common form, X-linked recessive (where the gene responsible for DC is carried on the X-chromosome). Early diagnosis and measurement of disease progress using the method of this invention is very beneficial for families with these genetic characteristics so that early treatment with anabolic steroids or bone-marrow-stimulating drugs can help prevent bone marrow failure. The non-invasive, patient friendly saliva-testing method of the present invention is particularly useful for DKC because babies and small children need testing and continued monitoring.

Idiopathic interstitial pneumonias are characterized by damage to the lung parenchyma by a combination of fibrosis and inflammation. Idiopathic pulmonary fibrosis (IPF) is an example of these diseases that causes progressive scarring of the lungs. Fibrous scar tissue builds up in the lungs over time, affecting their ability to provide the body with enough oxygen. Heterozygous mutations in the coding regions of the telomerase genes, TERT and TERC, have been found in familial and sporadic cases of idiopathic interstitial pneumonia. All affected patients with mutations have short telomeres. A significant fraction of individuals with IPF have short telomere lengths that cannot be explained by coding mutations in telomerase (Cronkhite, J. T., et al., Am. J. Resp. Crit. Care Med., 2008, 178:729-737). Thus, telomere shortening may be used as a marker for an increased predisposition toward this age-associated disease (Alder, J. K., et al., Proc. Natl. Acad. Sci. USA, 2008, 105(35):13051-13056). Further, the course of IPF varies from person to person. For some, the disease may progress slowly and gradually over years, while for others it may progress rapidly. The method of the present may be conveniently used to monitor the course of pulmonary fibrosis and taking appropriate interventional steps as described above.

Aplastic anemia is a disease in which bone marrow stops making enough red blood cells, white blood cells and platelets for the body. Any blood cells that the marrow does make are normal, but there are not enough of them. Aplastic anemia can be moderate, severe or very severe. People with severe or very severe aplastic anemia are at risk for life-threatening infections or bleeding. Patients with aplastic anemia carrying telomerase mutations have an increased risk of developing myelodysplasia. Telomerase deficiency may cause variable degrees of telomere shortening in hematopoietic stem cells and lead to chromosomal instability and malignant transformation (Calado, R. T. and Young, N. S., The Hematologist, 2010 world wide web URL hematology.org/Publications/Hematologist/2010/4849.aspx). Aplastic anemia patients with shorter telomeres have a lower survival rate and are much more likely to relapse after immunotherapy than those with longer telomeres. Scheinberg et al. (JAMA, 2010, 304(12):1358-1364) found that relapse rates dropped as telomere lengths increased. The group of patients with the shortest telomeres was also at greater risk for a conversion to bone marrow cancer and had the lowest overall survival rates. The method of the present invention may be used in aplastic anemia patients to monitor the risk of developing major complications so that the clinical management of an individual may be tailored accordingly.

### 5.4 Drug Responsiveness

In another embodiment, the present invention is useful in monitoring effectiveness of therapeutics or in screening for drug candidates affecting telomere length or telomerase activity. The ability to monitor telomere characteristics can provide a window for examining the effectiveness of particular therapies and pharmacological agents. The drug responsiveness of a disease state to a particular therapy in an individual may be determined by the method of the present invention. For example, the present invention finds use in monitoring the effectiveness of cancer therapy since the proliferative potential of cells is related to the maintenance of telomere integrity. As described above, while normal human somatic cells do not or only transiently express telomerase and therefore shorten their telomeres with each cell division, most human cancer cells typically express high levels of telomerase and show unlimited cell proliferation. Roth et al., (ibid., 2010) have suggested that individuals with cancer who have very short telomeres in their tumors (in which the shortest telomeres in most cells are near to telomere dysfunction) and high telomerase activity might benefit the most from anti-cancer telomerase-inhibiting drugs. Because telomerase is either not expressed or expressed transiently and at very low levels in most normal cells, telomerase inhibition therapies may be less toxic to normal cells than conventional chemotherapy. An example of such drugs is the short oligonucleotide-based telomerase inhibitor imetelstat (previously named GRN163L). Imetelstat is a novel lipid-based conjugate of the first-generation oligonucleotide GRN163 (Asai, A. et al., Cancer Res., 2003, 63:3931-3939). However, cancer patients having very short telomeres in normal blood cells (particularly their granulocytes) are at higher risk of adverse effects of imetelstat on proliferative tissues such as the bone marrow. Rattain et al. (2008) found that such subjects with short granulocyte TL were more likely to have bone marrow failure symptoms such as neutropenia or thrombocytopenia. In this situation, a doctor might prescribe a lower dose of imetelstat, a different drug, or more frequent monitoring for bone marrow problems.

In other embodiments, drug efficacy in the treatment of diseases of aging, for example but not limited to, cardiovascular disease, diabetes, pulmonary fibrosis, liver fibrosis, interstitial pneumonia and depression. In the case of cardiovascular disease, Brouilette et al. reported that middle-aged men with shorter telomere lengths than control groups benefit the most from lipid-lowering therapy with pravastatin (Brouilette, S. W. et al., Lancet, 2007, 369:107-114). Satoh et al. (Clin. Sci., 2009, 116:827-835) indicating that intensive lipid-lowering therapy protected telomeres from erosion better in patients treated with atorvastatin when compared with patients treated with moderate pravastatin therapy. The method of the present invention can be used to monitor the efficacy of statins in treated patients, wherein shorter telomere length correlates with better drug efficacy. Since subjects with the longest telomeres did not on average benefit from prophylactic statins, a doctor might suggest that the patient be especially compliant with good lifestyle habits as part of their treatment program. Conversely, patients with short telomeres who fear side effects of chronic statin usage might be persuaded to take statins based on their higher probability of benefiting from statins. Examples of statins that may be used include niacin (Advicor®, Simcor®), lovastatin (Altoprev®, Mevacor®), amolopidine (Caduet®), rosuvastatin (Crestor®), sitagliptin/simvastatin (Juvisync®), fluvastatin (Lescol®), pravastatin (Pravachol®), atorvastatin (Lipitor®), pitavastatin (Livalo®), and ezetimibe/simvastatin (Vytorin®).

In further embodiments, drug effectiveness in the treatment of telomeric diseases, for example but not limited to, Dyskeratosis congenita, pulmonary fibrosis, and aplastic anemia, may be measured. For example, dyskeratosis congenita and pulmonary fibrosis are both treated with high-dose steroids, which are well known to have numerous deleterious side effects. Use of the lowest possible steroid dose is thus highly desirable, making the method of the present invention a valuable tool for monitoring these patients.

### 5.5 Drug Candidate Screening

In another aspect, the present invention finds use as a general method of screening for candidate drugs affecting biological pathways regulating telomere length, such as telomerase activity. Ability to rapidly amplify telomere repeats provides a high thoroughput screening method for identifying small molecules, candidate nucleic acids, and peptides agents affecting telomere characteristics in a cell. Drug candidates that have a positive, telomere lengthening effect on normal cells would be preferred over those with telomere shortening (or telomerase inhibiting) effects, everything else being equal.

6. Methods

### 6.1 Laboratory Testing

The methods of this invention can be performed in a reference laboratory, e.g., a CLIA-certified laboratory. Samples containing subject DNA derived from saliva can be sent to the laboratory for testing. The results generated can be transmitted to a person or entity that ordered the test, or to the subject, themselves.

In a globalized economy there may be little geographic relationship between a location where a sample for testing is collected, a location of a person or entity who orders a test on the sample, a location where a test is performed, and a location where results of the test are received. Accordingly, a person or entity can transmit a sample to be tested from one location and receive a report including test results from a remote location, e.g., from a different city, state or country, transmitted by any method or means to access information. For example, results can be transmitted electronically. Results can be posted on the internet for access by, for example, a person ordering the test. Results can be pushed to a mobile and/or hand-held device, such as a smart phone.

### 6.2 In Vitro Diagnostic Kit

An in vitro diagnostic kit for telomere length assessment can include a tagged, e.g., bar-coded, saliva collection device and a pre-paid envelope for shipping the collected sample to a central testing facility. Alternatively, a point-of-care kit for use in doctors' offices can contain contains necessary reagents and solutions for purifying DNA and running the qPCR telomere assay on equipment in the doctor's office or non-laboratory setting. A related approach would be one in which the reagents and solutions were contained within an enclosed cassette or similar device for insertion into a closed, automated instrument that does both DNA purification and qPCR analysis. The output can be a test report with T/S value, a T-score for the subject's T/S value relative to a reference population, a Z-score for the subject's T/S value relative the age- and gender-matched normal population, and a clinical interpretation.

### EXAMPLES

### Example 1

### Device for Saliva Collection

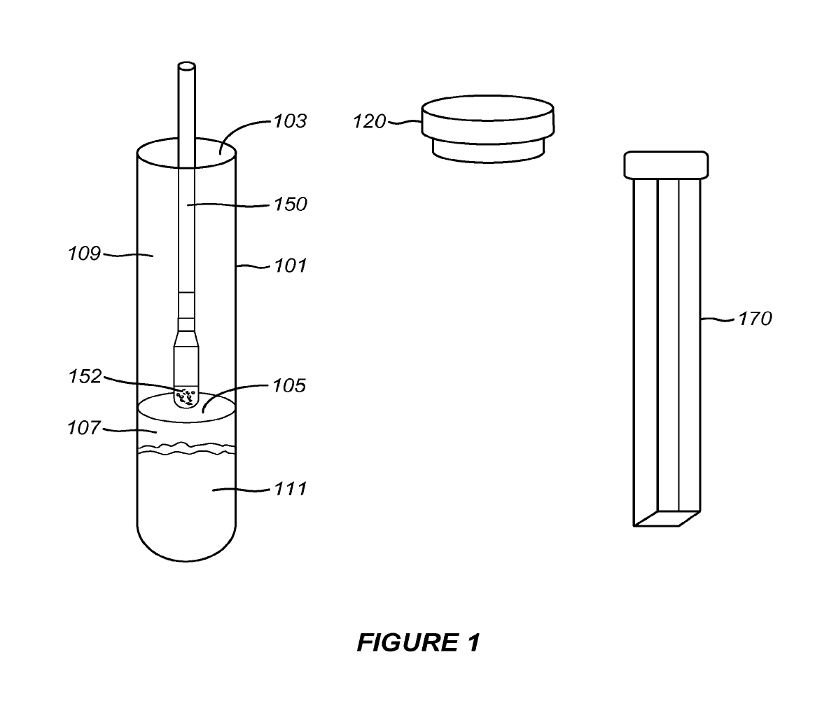

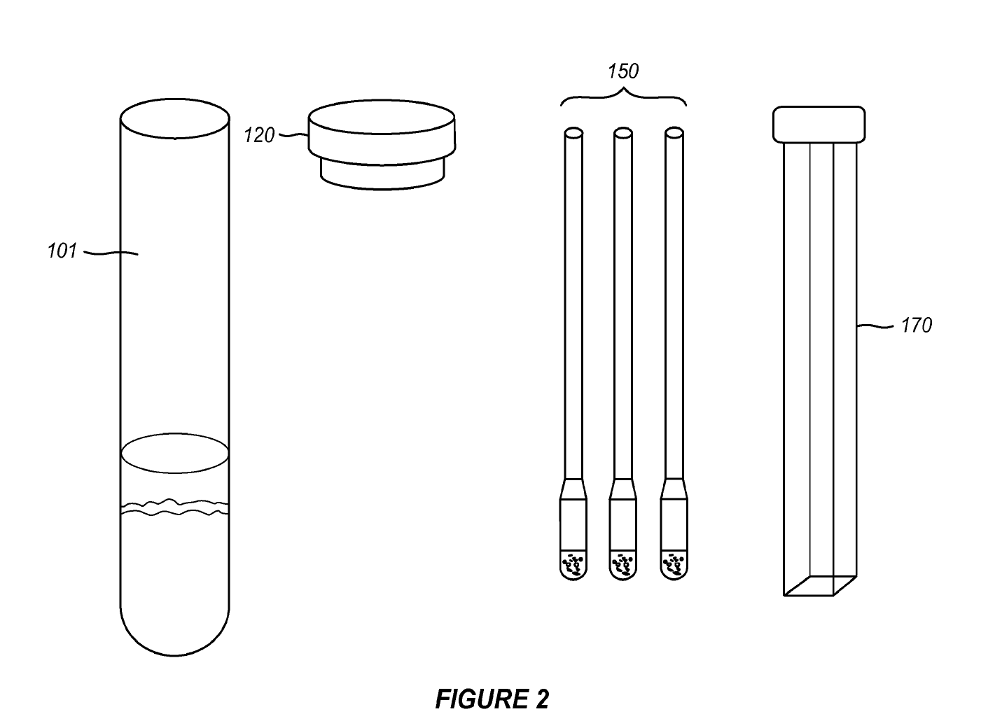

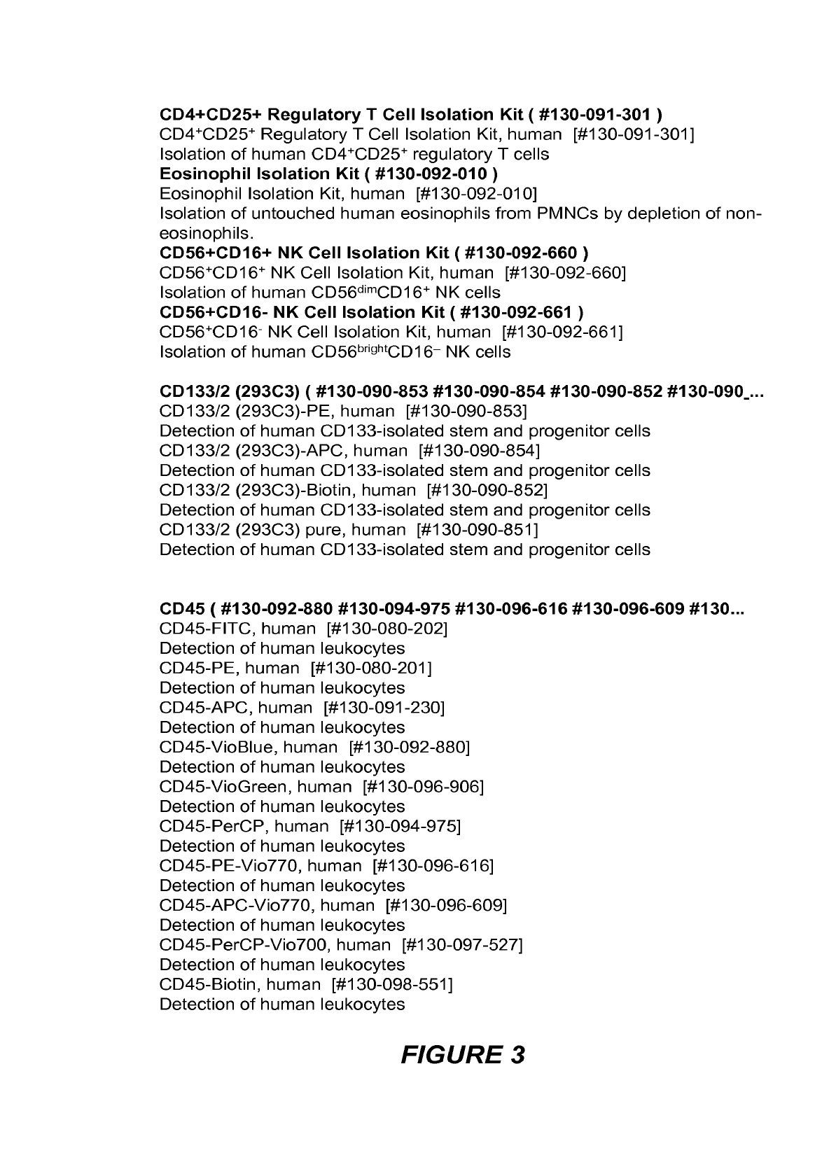

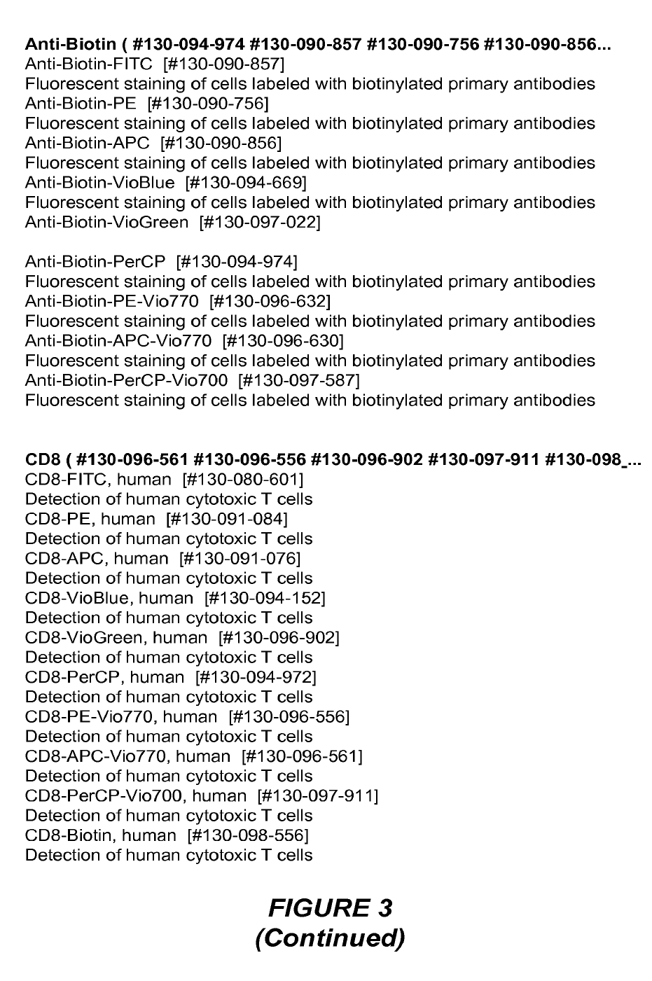

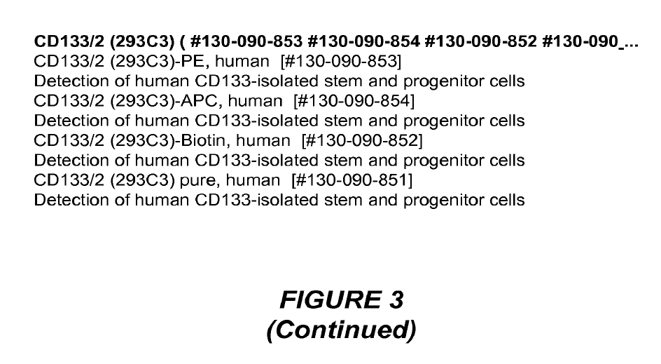

Referring to FIG. 1, a kit of this invention includes container 101 configured as a tube or a vial. Container 101 comprises a pliable material that can be compressed upon application of pressure, for example, manual pressure. Container 101 includes an opening 103 configured to accept a liquid sample containing biological material, such as a saliva sample. Container 101 also includes septum 105 that divides the container into fluidically isolated compartments, in this case, a closed compartment 107 and an open compartment 109 in communication with the opening. Closed compartment 107 contains a lysis buffer 111 configured to lyse cells in a saliva sample deposited in open compartment 109. The lysis buffer also contains chemicals to preserve nucleic acids. The kit also contains a screw top or snap cap 120 configured to seal opening 103.

The kit also includes a capture wand, 150. Capture wand 150 has an end, 152 that has attached to it antibodies that selectively bind EpCAM, a molecule on the surface of epithelial cells. Capture wand 150 does not have capture moieties that bind either myeloid cells or lymphoid cells. The kit also includes wand container 170 configured to hold and store capture wand 150 after use. Container 170 may contain a stabilizing/denaturing solution similar to the solution in compartment 111.

Before use, a subject can clean his or her mouth, for example by flossing, brushing and/or using mouthwash.

In use, a subject spits or drools saliva into open compartment 109, where it is separated from the lysis buffer in closed compartment 107 by septum 105. The subject may also be instructed to add a solution designed to reduce viscosity into compartment 109 to facilitate binding of the target cells to the binding agent. The subject inserts capture wand 150 into the collected saliva and captures epithelial cells on it, for example by swirling or stirring. Capture wand 150 remains in contact with the saliva sample for about 10 seconds. Then, capture wand 150 is removed from container 101, removing epithelial cells with it and enriching the saliva sample for myeloid and lymphoid cells. Capture wand 150 is inserted into wand container 170 for subsequent use. Alternatively, capture wand 150 can be discarded.

After removing capture wand 150 from container 101, the subject closes container 101 with snap cap 120. The subject squeezes container 101 to break septum 105 and put the saliva sample in contact with lysis buffer 111. The subject mixes the saliva sample with lysis buffer 111, for example by shaking or swirling.

The subject can now send closed container 101 and/or wand container 170 to a service provider for analysis.

### Example 2

### Determination of Telomeric Length by qPCR

This method is adapted from the published original qPCR method of Cawthon (Nucleic Acid Res., 2002, 30(10):e47) combined with the analysis method of Blackburn et al (Lin, J. et al., J. Immunol. Methods, 2009). The assay consists of two separate PCR reactions: A T (telomeric) PCR value is obtained along with an S (single copy gene, for example beta-globin) value and the two values are compared. The T value is divided by the S value to determine the relative length of a sample telomere per genome.

Subject saliva is collected using a kit of this invention or, e.g., an Oragene® Genotek DNA kit (DNA Genotek Inc., Kanata, Ontario, Canada), which contains a protease and denaturing solution that lyses the cells present, inhibits enzymes that might degrade nucleic acids, and thus stabilizes the genomic DNA present. Although no further purification is technically needed to generate T/S values, it has been found that DNA purity can affect T/S ratios, potentially due to contaminants that affect one or more qPCR reaction steps. Thus, one method includes a DNA purification step as described above. A DNA reference standard (e.g., HeLa DNA or an NIST standard) for determining concentrations, a single or set of normalizing controls to adjust for shifts in the assay over time, and QC controls for assay acceptance are included with each assay.

Real time quantitative PCR on the denatured DNA samples is performed on using 384-well plates so that subject samples and control samples may be run on the same plate. Two master mixes of PCR reagents are prepared, one with the telomere (T) primer pair, the other with the single copy gene (S) primer pair. The T primer pair is used at a final concentration of 100 nM and has the following sequences: tel 1b 5′-CGGTTTGTTTGGGTTTGGGTTTGGGTTTGGGTTTGGGTT-3′ [SEQ. ID No.: 3]; and tel 2b 5′-GGCTTGCCTTACCCTTACCCTTACCCTTACCCTTACCCT-3′ [SEQ. ID No.: 4]. The S primer pair, derived from human beta-globin, is used at a final concentration of 300 nm and has the following sequences: hbg 1 5′-GCTTCTGACACAACTGTGTTCACTAGC-3′ [SEQ. ID No.: 5]; and hbg 2 5′-CACCAACTTCATCCACGTTCACC-3′ [SEQ. ID No.: 6]. The primers are obtained from Integrated DNA Technologies (Coralville, Iowa) as standard-purified material.

The plates are carefully labeled to identify subject samples wells and standard wells. On separate plates, 7.5 ul of either T master mix or S master mix is added each sample and standard curve well. For each subject in whom the T/S ratio is assayed, three identical 3.5 ul aliquots of the DNA sample are added to the appropriately labeled wells. To obtain a standard curve, one standard HeLa DNA sample is diluted serially in 10×PCR buffer (200 mM Tris-HCl, pH 8.4; 500 mM KCl) to produce five concentrations of DNA (26, 8.75, 2.9, 0.97, 0.324 and 0.108 ng). PCR reagents are then added to each well such that the final reaction mixture in each well contains 20 mM Tris-HCl, pH 8.4; 50 mM KCl; 200 uM each dNTP (Roche Applied Science); 1% DMSO; 0.4× Syber Green I (Invitrogen); and 0.4 Units of Platinum Taq DNA polymerase (Invitrogen) per 11 ul reaction. Subject sample and normalizing control wells additionally contain 0.5-10 ng of genomic DNA. QC negative control wells additionally contain 22 ng E. coli DNA.

DNA amplification is then carried out on a Roche Lightcyler™ (Roche Applied Science) instrument. T (telomic) DNA is denatured at 96° C. for 1 sec, followed by annealing and extension at 54° C. for 60 sec. S (single copy gene) DNA is denatured at 95° C. for 15 sec, annealed at 58° C. for 1 sec, extended at 72° C. for 20 sec (8 cycles), denatured at 96° C. for 1 sec, annealed at 58° C. for 1 sec, extended at 72° C. for 20 sec, and held at 83° C. for 5 sec with data collection (35 cycles).

The LightCycler software is used to generate the standard curve for each plate and to analyze the data for outliers that are discarded. A raw T/S ratio (T mean conc./S mean conc.) is calculated for each subject sample. An adjustment factor is calculated by dividing the mean T/S ratio of the five normalizing control samples by the standard T/S values. The average percent difference for these five values is used as the adjustment factor. Each raw T/S ratio is divided the adjustment factor to give the final T/S value for each sample, which is the telomere length. Final T/S values are also calculated for each low, medium and high Quality Control and compared to acceptable ranges.

### Example 3

### Determination of Cardiovascular Risk Based on Telomere Length

Typically T/S values are determined in a large population of defined age ranges for individuals at low or moderate risk for cardiovascular disease (“CVD”). Data on a variety of standard risk biomarkers and factors that might co-vary with telomere length might also be collected at baseline. The subjects are then followed for a period of time (e.g. 5-15 years) and data on incidence of cardiovascular events, including death, are collected. The study might be designed to include a treatment group (e.g., prophylactic statins as in Brouilette et al., 2007) as part of a randomized, placebo controlled study. Subjects are binned into groups representing the baseline top tertile (33%) who have the longest telomeres, the mid tertile, and the lowest tertile. Other quintiles (e.g. quartiles) might be used instead of tertiles. At the end of the study, odds ratios for an event as a function of telomere length can then be calculated by comparing the number of events in the lowest or mid tertile group to the number of events in the highest tertile group (the reference group).

In several studies using the qPCR technology for T/S determination, the tertile boundaries in studies involving middle-aged individuals have been found to be roughly 0.9 and roughly 1.3, indicating subjects with T/S less than 0.9, or 0.9-1.3, or greater than 1.3 are relatively speaking at high, moderate, and low risk of disease. The exact conditions used in running the qPCR assay can influence T/S ratios, hence each lab typically reports the thresholds for the boundaries between quintiles specific for their methodology. By including assessment of standard risk factors for CVD (e.g. lipid profile, family history, hsCRP, blood pressure, glucose and insulin, etc.) statisticians can use multivariate analysis to determine whether TL is an independent risk factor for disease, and the relative utility and significance of TL versus other risk factors. In both the Brouilette and Willeit publications, TL was a strong and independent risk factor for CVD, even with the most aggressive multivariate models.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-69. (canceled)

70. A kit comprising:
(a) a container having an opening adapted to receive a liquid sample through the opening;
(b) a cover configured to reversibly seal the opening; and
(c) a capture device configured to be introduced into the container, wherein the capture device is configured to selectively bind cells of a first type and not to substantially bind cells of a second cell type.

71. The kit of claim 70, wherein the capture device substantially binds at least one cell other than a lymphoid and/or myeloid cell.

72. The kit of claim 70, wherein the cells of a first type are targeted for removal and the cells of a second type are targeted for analysis.

73. The kit of claim 70, wherein the cells of the first type comprise epithelial cells and the cells of the second type comprise at least one of myeloid cells and lymphoid cells.

74. The kit of claim 70, wherein the capture device binds a surface molecule on epithelial cells.

75. The kit of claim 74, wherein the surface molecule is Ep-CAM.

76. The kit of claim 70, wherein the container includes a first compartment and a second compartment, wherein:
a) the first compartment and the second compartment are separated from one another by a breakable barrier;
b) the first compartment is configured to receive a liquid sample from the opening in the container;
c) the second compartment is closed and contains a cell lysis solution; and
wherein the container is configured such that breaking the breakable barrier mixes a sample solution in the first compartment with the lysis solution in the second compartment.

77. The kit of claim 76, wherein the container comprises a vial having an open end and a closed end, wherein the first compartment is positioned closer to the open end and the second compartment is positioned closer to the closed end, and wherein the breakable barrier comprises a septum in the vial.

78. The kit of claim 76, wherein the capture device is configured as a wand comprising a collar, wherein the collar is configured to prevent a tip of the wand from piercing the breakable barrier when the wand is inserted into the opening of the container.

79. The kit of claim 76, wherein the container is configured such that manual squeezing of the container breaks the breakable barrier.

80. The kit of claim 76, wherein the first compartment comprises a non-denaturing solution.

81. The kit of claim 73, further comprising a container for the capture device.

82. The kit of claim 70, further comprising a second capture device comprising second binding moieties that bind cells other than epithelial cells and that do not substantially bind myeloid cells or lymphoid cells.

83. A method of determining a measure of telomere abundance, the method comprising:
a) contacting a saliva sample with capture particles in a container, wherein the capture particles have moieties that bind epithelial cells;
b) removing the epithelial cells present in the saliva sample from the other cells present in the saliva sample, wherein the other cells present in the saliva sample include at least one cell type selected from myeloid cells and lymphoid cells;
c) isolating DNA from the other cells present in the saliva sample; and
d) determining a measure of telomere abundance present in the other cells by amplifying the DNA.

84. The method of claim 83, wherein the sample comprises saliva enriched for lymphoid and/or myeloid cells.

85. The method of claim 83, wherein telomere abundance is a measure of relative abundance.

86. The method of claim 85, wherein the measure of relative abundance is abundance of telomeric DNA relative to abundance of total sample genomic DNA.

87. The method of claim 86, wherein the measure of relative abundance is abundance of telomeric DNA relative to abundance of a genomic reference sequence.

88. The method of claim 87, wherein the genomic reference sequence is a single copy reference nucleotide sequence (e.g., human beta-globin) or abundance of non-telomere repetitive DNA (e.g., Alu repeats or centromeric repeats).


SEQUENCE LISTING (7 sequences)

SEQ ID NO: 1 - DNA, 37 bp, Artificial Sequence
  synthetic construct; primer
ggtttttgagggtgagggtgagggtgagggtgagggt

SEQ ID NO: 2 - DNA, 39 bp, Artificial Sequence
  synthetic construct; primer
tcccgactatccctatccctatccctatccctatcccta

SEQ ID NO: 3 - DNA, 39 bp, Artificial Sequence
  synthetic construct; primer
cggtttgtttgggtttgggtttgggtttgggtttgggtt

SEQ ID NO: 4 - DNA, 39 bp, Artificial Sequence
  synthetic construct; primer
ggcttgccttacccttacccttacccttacccttaccct

SEQ ID NO: 5 - DNA, 27 bp, Artificial Sequence
  synthetic construct; primer
gcttctgacacaactgtgttcactagc

SEQ ID NO: 6 - DNA, 23 bp, Artificial Sequence
  synthetic construct; primer
caccaacttcatccacgttcacc

SEQ ID NO: 7 - DNA, 18 bp, Artificial Sequence
  synthetic construct; probe
aatcccaatcccaatccc